(19) United States
(12) Patent Application Publication
(10) Pub. No.: US 2024/0217997 A1
(43) Pub. Date: Jul. 4, 2024

(54) NOVEL MEPICIDES AS ANTIMICROBIAL AGENTS

(71) Applicant: THE GEORGE WASHINGTON UNIVERSITY, NW Washington, DC (US)

(72) Inventors: Cynthia S. DOWD, NW Washington, DC (US); Darean BAGUE, Arlington, VA (US); Ruiqin WANG, Annandale, VA (US); Audrey R. Odom JOHN, Philadelphia, PA (US); Robin D. COUCH, Manassas, VA (US); Helena I. BOSHOFF, Potomac, MD (US)

(21) Appl. No.: 18/555,194

(22) PCT Filed: Apr. 15, 2022
(86) PCT No.: PCT/US2022/024966

§ 371 Date: Oct. 12, 2023

Related U.S. Application Data
(60) Provisional application No. 63/175,444, filed on Apr. 15, 2021.

(51) Int. Cl. C07F 9/40; A61K 31/662

(52) U.S. Cl. CPC C07F 9/4056; A61K 31/662

(57) ABSTRACT

The present disclosure relates to novel compounds useful as antimicrobial agents. The present disclosure also relates to processes for their preparation, pharmaceutical compositions comprising them, and to their use in methods for treating or preventing microbial infections caused by parasites or bacteria, such as, for example, Plasmodium falciparum or related Plasmodium parasite species, Mycobacterium tuberculosis or related Mycobacterium bacteria species, S. aureus, and ESKAPE pathogens.

### CROSS REFERENCE TO RELATED APPLICATIONS

This application claims the benefit of U.S. Provisional Application No. 63/175,444, filed Apr. 15, 2021, the entire contents of which are hereby incorporated by reference.

### STATEMENT REGARDING FEDERALLY SPONSORED RESEARCH OR DEVELOPMENT

At least some aspects of this invention were made with Government support from National Institutes of Health under Grant No. A1 123433. The Government may have certain rights in this invention.

### FIELD OF THE INVENTION

The present invention relates to novel compounds useful as antimicrobial agents. The present invention also relates to processes for their preparation, pharmaceutical compositions comprising them, and to their use in methods for treating or preventing microbial infections caused by parasites or bacteria, such as, for example, Plasmodium falciparum or related Plasmodium parasite species, Mycobacterium tuberculosis or related Mycobacterium bacteria species, S. aureus, and ESKAPE pathogens, including drug resistant strains of such microorganisms.

### BACKGROUND OF THE INVENTION

Despite intense efforts in drug development and aggressive vector control programs, malaria remains a formidable challenge to public health. According to recent estimates, malaria causes 212 million clinical cases and more than 429,000 deaths each year, predominately in young children living in sub-Saharan Africa. While 5 species of Apicomplexan parasites of the genus Plasmodium cause human malaria, Plasmodium falciparum is the most deadly. Due to pervasive drug resistance, P. falciparum treatment has become increasingly dependent on a single class of compounds, the artemisinins. However, there is substantial evidence to suggest that the effectiveness of artemisinin combination therapies (ACTs) is waning, and as such, global malaria control efforts are threatened. The rapid increase in multidrug-resistant parasites combined with a chronic under-investment in drug discovery has severely limited existing therapies. As only a few new antimalarial agents are in the clinical pipeline, identification of novel drug targets is essential.

The methylerythritol phosphate (MEP) pathway of isoprenoid biosynthesis is an unexploited drug target present in most eubacteria and apicomplexan protozoa. In P. falciparum, the MEP pathway enzymes are apicoplast-localized, and data suggest that isoprenoid precursor biosynthesis is the only essential function of the plastid organelle in blood-stage parasites. The pathway begins with the condensation of pyruvate and glyceraldehyde-3-phosphate and then proceeds through a series of enzymatic reactions to produce isopentenyl pyrophosphate (IPP) and dimethylallyl diphosphate (DMAPP), which are used to synthesize downstream products. The enzymes of the MEP pathway are essential, as isoprenoids are required for numerous cellular processes including aerobic respiration, membrane stability, and protein prenylation. Importantly, humans employ an alternate route for isoprenoid generation, using instead the mevalonate pathway whose components lack similarity to MEP pathway enzymes. Due to the essentiality of the MEP pathway in P. falciparum (FIG. 1) and the absence of mammalian homologs, compounds that would specifically inhibit enzymes in the pathway are paramount.

The first committed enzyme of the MEP pathway is catalyzed by 1-deoxy-D-xylulose-5-phosphate reductoisomerase (Dxr/IspC, EC 1.1.1.267), and considerable efforts have been made to effectively target the enzyme. Dxr catalyzes the reductive isomerization of 1-deoxy-D-xylylose 5-phosphate (DOXP) to 2-C-methyl-D-erythritol 3-phosphate (MEP), using a divalent cation (Mg²⁺, Mn²⁺, or Co²⁺) and NADPH as a cofactor. Chemical inhibition of Dxr in blood-stage P. falciparum depletes cellular MEP metabolites, and ultimately kills the parasites. Moreover, genetic disruption of the Dxr locus in P. falciparum (PF3D7 1467300) is only feasible if cultures are artificially supplemented with downstream isoprenoids. Further, Dxr is druggable, contains a high flux-control coefficient, and is one of only seven antimalarial targets that have been clinically validated.

Mycobacterium tuberculosis (Mtb) is the causative agent of tuberculosis. Two mechanisms are known for the biosynthetic production of isoprenoid units: the mevalonate pathway found in mammals and plants, and the nonmevalonate pathway found in most bacteria. There are no human homologues for the enzymes of the nonmevalonate pathway and each enzymatic reaction is vital to the survival of bacteria. These enzymes are thus prospective targets for therapeutic intervention of M. tuberculosis. Dxr is essential for the growth of Mtb. Current anti-TB drugs do not target the nonmevalonate pathway, so Dxr inhibition would be a new mechanism of action.

Fosmidomycin (1a), isolated from Streptomyces lavendulae, is a potent inhibitor of P. falciparum DXR (IC50=0.034 uM). FR900098 (1b), the N-acetyl analog of fosmidomycin isolated from Streptomyces rubellomurinus, is roughly equipotent to fosmidomycin (P. falciparum DXR IC₅₀=0.024 μM). While these two natural products have submicromolar inhibition of P. falciparum growth (IC₅₀=0.09-0.35 μM), their use as a single drug therapy is limited by low bioavailability, short serum half-life, and malaria recrudescence.

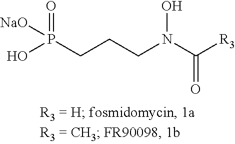

There is therefore a need for new Dxr inhibitors to combat microbial infections caused by, for example, P. falciparum malaria and M. tuberculosis.

### SUMMARY OF THE INVENTION

In one aspect, the present invention relates to a compound of formula (I)

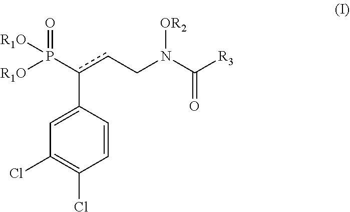

or a tautomer thereof, stereoisomer thereof, prodrug thereof, or pharmaceutically acceptable salt thereof,
wherein
---- represents a bond or is absent;
each R₁ is, independently, —NH₄, —N(alkyl)₄, —N(aryl)₄, C₁₋₄ alkyl (e.g., —CH₂CH₃), or —(CR^{a}R^{b})ₘ—O(C═O)—C₁₋₆ alkyl (e.g., —CH₂—O—C(═O)—C(CH₃)₃), wherein the atom at the left is attached to the oxygen atom;
R₂ is H, —(CR^{c}R^{d})ₙ-aryl, or —(CR^{c}R^{d})ₙ-heteroaryl, wherein the atom at the left is attached to the oxygen atom;
R₃ is H, C₁₋₆ alkyl (e.g., methyl), C₁₋₆ haloalkyl, C₃₋₆ cycloalkyl, C₁₋₆ alkoxy, C₁₋₆ haloalkoxy, C₃₋₆ cycloalkoxy, —(CR_{c}R_{d})ₚ-aryl or or —(CR^{c}R^{d})ₙ-heteroaryl;
each of R^{a}, R^{b}, R^{c}, and R^{d} is independently H, halogen, or C₁₋₄ alkyl (e.g. methyl, or ethyl);
each of m and n is independently 1, 2, 3, or 4; and
p is 0, 1, 2, 3, or 4;
wherein each aryl or heteroaryl is, independently, optionally substituted with up to five R⁴ selected from the group consisting of halogen, hydroxyl, cyano, amino, (C₁₋₆ alkyl)amino, di(C₁₋₆ alkyl)amino, C₁₋₆ alkyl, C₁₋₆ haloalkyl, C₃₋₆ cycloalkyl, C₁₋₆ alkoxy, C₁₋₆ haloalkoxy, C₃₋₆ cycloalkoxy, arylalkyl (e.g., benzyl) and heteroaryl (e.g., pyridine, furan, thiophene, indole);
with the proviso that when ---- is absent, each R₁ is ethyl or each R₁ is —CH₂—O—C(═O)—C(CH₃)₃, and R₃ is methyl, then R₃ is not

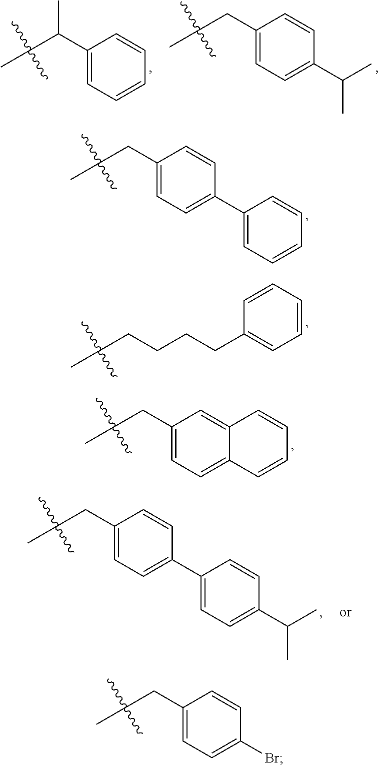

where the squiggly line ( [symbol] ) represents the point of attachment of R₂ to the reset of the molecule.

In another aspect, the present invention relates to a pharmaceutical composition comprising a compound as disclosed in any embodiment herein and a pharmaceutically acceptable excipient.

In another aspect, the present invention relates to a method for treating or preventing a microbial infection in a subject (e.g., a subject in need thereof) comprising administering to the subject an effective amount of a compound as disclosed in any embodiment herein. In some embodiments, the microbial infection is malaria. In some embodiments, the microbial infection is tuberculosis.

In another aspect, the present invention relates to a method for treating or preventing a pathogen in a subject (e.g., a subject in need thereof) comprising administering to the subject an effective amount of a compound as disclosed in any embodiment herein. In some embodiments, the pathogen is Plasmodium falciparum or related Plasmodium parasite species, Mycobacterium tuberculosis or related Mycobacterium bacteria species, S. aureus, or ESKAPE pathogen.

### BRIEF DESCRIPTION OF THE DRAWINGS

FIG. 1 depicts the methyl erythritol phosphate (MEP) pathway of isoprenoid biosynthesis.

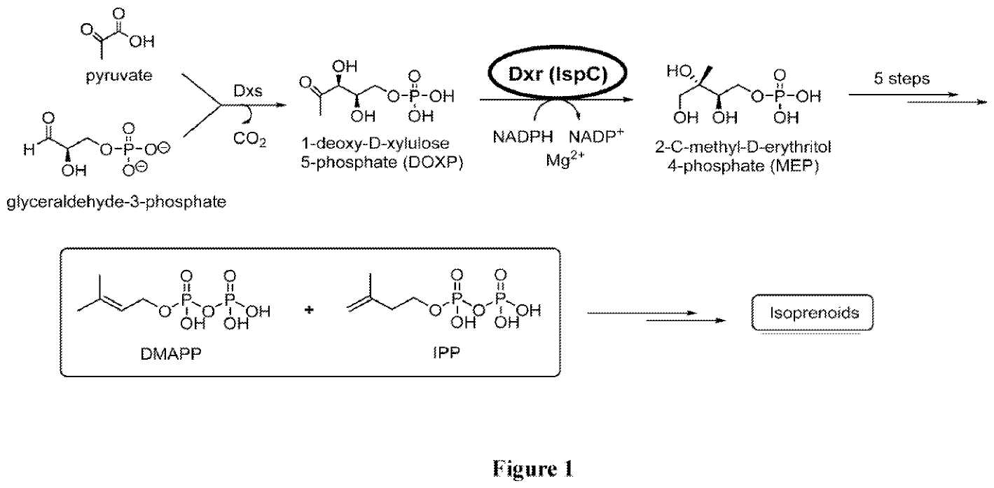

FIG. 2 depicts exemplary Mycobacterium tuberculosis whole cell minimum inhibitory concentration (MIC) data (μg/ml) for fosmidomycin (FSM), Compound A and Compound B (see Table 2).

### DETAILED DESCRIPTION OF THE INVENTION

As used herein the following definitions shall apply unless otherwise indicated.

The term “in need thereof’ refers to a subject infected with a microbial pathogen or at risk of becoming infected by the microbial pathogen. In some cases, the microbial pathogen is a eukaryotic pathogen, and more specifically a eukaryotic pathogen belonging to the genus Plasmodium. In some cases the pathogen is a prokaryotic pathogen, and more specifically belonging to the genus Mycobacterium.

As used throughout, the phrase an “effective amount” of a compound of this disclosure is measured by the therapeutic effectiveness of the compound, wherein at least one adverse effect of a disorder is ameliorated or alleviated. More specifically, administering a compound or composition results in complete or at least partial inhibition of a metabolic pathway or other biological processes in a pathogen. In addition, an effective amount is sufficient to result in at least some degree of alleviation or prevention of an infection caused by a pathogen, or prevention of an infection by the pathogen.

The terms “treating or preventing” are intended to include preventing, eradicating, or inhibiting the resulting increase of undesired physiological activity associated with a disorder or infection, for example, in the context of the therapeutic or prophylactic methods of the invention. In another embodiment, the term treating or preventing includes antagonistic effects, e.g., diminishment of the activity or production of mediators of a disorder.

As used herein and unless otherwise indicated, the term “formulation” refers to a composition comprising a compound of the present disclosure that is described in a particular dosage form (e.g., tablet) or with a particular dosage amount.

When administered to a subject (e.g., to an animal for veterinary use or to a human for clinical use), the compounds of the invention can be optionally administered in isolated form.

The phrase “pharmaceutically acceptable salt(s),” as used herein includes but is not limited to salts of acidic or basic groups that may be present in compounds of the present disclosure. Compounds in the present disclosure that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions including, but not limited to, sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds in the present disclosure that include an amino moiety may form pharmaceutically acceptable salts with various amino acids, in addition to the acids mentioned above. Compounds in the present disclosure that are acidic in nature are capable of forming base salts with various pharmacologically acceptable cations. Examples of such salts include alkali metal or alkaline earth metal salts and ammonium salts, for example, calcium, magnesium, sodium, potassium, lithium, zinc, potassium, and iron salts.

As used herein and unless otherwise indicated, the terms “prodrug” or “pharmaceutically acceptable prodrug” means a derivative of a compound that can hydrolyze, oxidize, or otherwise react under biological conditions (in vitro or in vivo) to provide the compound. Examples of prodrugs include, but are not limited to, compounds that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include compounds that comprise oligonucleotides, peptides, lipids, aliphatic and aromatic groups, or NO, NO₂, ONO, and ONO₂ moieties. Prodrugs can typically be prepared using well known methods, such as those described in Burger's Medicinal Chemistry and Drug Discovery, pp. 172, 178, 949, 982 (Manfred E. Wolff ed., 5th ed. 1995), and Design of Prodrugs (H. Bundgaard ed., Elselvier, New York 1985).

The phrase “pharmaceutically acceptable excipient” may be any substance, not itself a therapeutic agent, used as a carrier, diluent, adjuvant, binder, and/or vehicle for delivery of a therapeutic agent to a patient, or added to a pharmaceutical composition to improve its handling or storage properties or to permit or facilitate formation of a compound or pharmaceutical composition into a unit dosage form for administration. Pharmaceutically acceptable excipients are known in the pharmaceutical arts and are disclosed, for example, in Remington: The Science and Practice of Pharmacy, 21st Ed. (Lippincott Williams & Wilkins, Baltimore, MD, 2005). As will be known to those in the art, pharmaceutically acceptable excipients can provide a variety of functions and can be described as wetting agents, buffering agents, suspending agents, lubricating agents, emulsifiers, disintegrants, absorbents, preservatives, surfactants, colorants, flavorants, and sweeteners.

In the present disclosure, the term “halo” or “halogen” as used by itself or as part of another group refers to —Cl, —F, —Br, or —I. In one embodiment, the halo is —Cl or —F. In one embodiment, the halo is —Cl.

In the present disclosure, the term “nitro” as used by itself or as part of another group refers to —NO₂.

In the present disclosure, the term “cyano” as used by itself or as part of another group refers to —CN.

In the present disclosure, the terms “hydroxy” and “hydroxyl” as used by itself or as part of another group refers to —OH.

In the present disclosure, the term “alkyl” as used by itself or as part of another group refers to unsubstituted straight- or branched-chain aliphatic hydrocarbons containing from one to twelve carbon atoms, i.e., C₁₋₁₂ alkyl, or the number of carbon atoms designated, e.g., a C1 alkyl such as methyl, a C₂ alkyl such as ethyl, a C₃ alkyl such as propyl or isopropyl, a C1-₃ alkyl such as methyl, ethyl, propyl, or isopropyl, and so on. In one embodiment, the alkyl is a C₁₋₁₀ alkyl. In another embodiment, the alkyl is a C₁₋₆ alkyl. In another embodiment, the alkyl is a C₁₋₄ alkyl. In another embodiment, the alkyl is a straight chain C₁₋₁₀ alkyl. In another embodiment, the alkyl is a branched chain C₃₋₁₀ alkyl. In another embodiment, the alkyl is a straight chain C₁₋₆ alkyl. In another embodiment, the alkyl is a branched chain C₃₋₆ alkyl. In another embodiment, the alkyl is a straight chain C₁₋₄ alkyl. In another embodiment, the alkyl is a branched chain C₃₋₄ alkyl. In another embodiment, the alkyl is a straight or branched chain C₃₋₄ alkyl. Non-limiting exemplary C₁₋₁₀ alkyl groups include methyl, ethyl, propyl, isopropyl, butyl, sec-butyl, tert-butyl, iso-butyl, 3-pentyl, hexyl, heptyl, octyl, nonyl, and decyl. Non-limiting exemplary C₁₋₄ alkyl groups include methyl, ethyl, propyl, isopropyl, butyl, sec-butyl, tert-butyl, and iso-butyl.

In the present disclosure, the term “cycloalkyl” as used by itself or as part of another group refers to saturated and partially unsaturated (containing one or two double bonds) cyclic aliphatic hydrocarbons containing one to three rings having from three to twelve carbon atoms, i.e., C₃₋₁₂ cycloalkyl. or the number of carbons designated. In one embodiment, the cycloalkyl group has two rings. In one embodiment, the cycloalkyl group has one ring. In another embodiment, the cycloalkyl group is chosen from a C₃₋₈ cycloalkyl group. In another embodiment, the cycloalkyl group is chosen from a C cycloalkyl group. Non-limiting exemplary cycloalkyl groups include cyclopropyl, cyclobutyl, cyclopentyl, cyclohexyl, cycloheptyl, cyclooctyl, norbornyl, decalin, adamantyl, cyclohexenyl, and cyclopentenyl, cyclohexenyl.

In the present disclosure, the term “alkenyl” as used by itself or as part of another group refers to an alkyl group as defined above containing one, two or three carbon-to-carbon double bonds. In one embodiment, the alkenyl group is chosen from a C₂₋₆ alkenyl group. In another embodiment, the alkenyl group is chosen from a C₂₋₄ alkenyl group. Non-limiting exemplary alkenyl groups include ethenyl, propenyl, isopropenyl, butenyl, sec-butenyl, pentenyl, and hexenyl.

In the present disclosure, the term “alkynyl” as used by itself or as part of another group refers to an alkyl group as defined above containing one to three carbon-to-carbon triple bonds. In one embodiment, the alkynyl has one carbon-to-carbon triple bond. In one embodiment, the alkynyl group is chosen from a C₂₋₆ alkynyl group. In another embodiment, the alkynyl group is chosen from a C₂₋₄ alkynyl group. Non-limiting exemplary alkynyl groups include ethynyl, propynyl, butynyl, 2-butynyl, pentynyl, and hexynyl groups.

In the present disclosure, the term “haloalkyl” as used by itself or as part of another group refers to an alkyl group substituted by one or more fluorine, chlorine, bromine and/or iodine atoms. In one embodiment, the alkyl group is substituted by one, two, or three fluorine and/or chlorine atoms. In another embodiment, the haloalkyl group is a C₁₋₆ haloalkyl group In another embodiment, the haloalkyl group is a C₁₋₄ haloalkyl group. Non-limiting exemplary haloalkyl groups include fluoromethyl, 2-fluoroethyl, difluoromethyl, trifluoromethyl, pentafluoroethyl, 1,1-difluoroethyl, 2,2-difluoroethyl, 2,2,2-trifluoroethyl, 3,3,3-trifluoropropyl, 4,4,4-trifluorobutyl, and trichloromethyl groups.

In the present disclosure, the term “alkoxy” as used by itself or as part of another group refers to an optionally substituted alkyl, optionally substituted cycloalkyl, optionally substituted alkenyl or optionally substituted alkynyl attached to a terminal oxygen atom. In one embodiment, the alkoxy group is chosen from a C₁₋₄ alkoxy group. In another embodiment, the alkoxy group is chosen from a C₁₋₆ alkoxy group. In another embodiment, the alkoxy group is chosen from a C₁₋₄ alkyl attached to a terminal oxygen atom, e.g., methoxy, ethoxy, and tert-butoxy.

In the present disclosure, the term “haloalkoxy” as used by itself or as part of another group refers to a C₁₋₄ haloalkyl attached to a terminal oxygen atom. Non-limiting exemplary haloalkoxy groups include fluoromethoxy, difluoromethoxy, trifluoromethoxy, and 2,2,2-trifluoroethoxy.

In the present disclosure, the term “aryl” as used by itself or as part of another group refers to a monocyclic, bi cyclic, or tricyclic aromatic ring system having from six to fourteen carbon atoms, i.e., C₆-C₁₄ aryl. Non-limiting exemplary aryl groups include phenyl (abbreviated as “Ph”), 1-naphthyl, 2-naphthyl, phenanthryl, anthracyl, indenyl, azulenyl, biphenyl, biphenylenyl, and fluorenyl groups. In one embodiment, the aryl group is chosen from phenyl, 1-naphthyl, or 2-naphthyl. In one embodiment, the aryl is a bicyclic or tricyclic C₁₀-C₁₄ aromatic ring system.

The term “heteroaryl”, unless otherwise specified, refers to an optionally substituted 5-to-14-member aromatic ring having one or more heteroatoms selected from N, O, and S as ring atoms. The heteroaryl may be a mono-, bi- or tricyclic ring system. Examples of such “heterocyclic ring” or “heteroaryl” radicals include, but are not limited to, oxazolyl, thiazolyl, imidazolyl, pyrrolyl, furanyl, pyridinyl, pyrimidinyl, pyrazinyl, benzofuranyl, indolyl, benzothiazolyl, benzoxazolyl, carbazolyl, quinolyl, isoquinolyl, azetidinyl, acridinyl, benzodioxolyl, benzodioxanyl, benzofuranyl, carbazolyl, cinnolinyl, dioxolanyl, indolizinyl, naphthyridinyl, perhydroazepinyl, phenazinyl, phenothiazinyl, phenoxazinyl, phthalazinyl, pteridinyl, purinyl, quinazolinyl, quinoxalinyl, tetrazoyl, tetrahydroisoquinolyl, piperidinyl, piperazinyl, 2-oxopiperazinyl, 2-oxopiperidinyl, 2-oxopyrrolidinyl, 2-oxoazepinyl, azepinyl, 4-piperidonyl, pyrrolidinyl, pyridazinyl, oxazolinyl, oxazolidinyl, triazolyl, indanyl, isoxazolyl, isoxazolidinyl, morpholinyl, thiazolinyl, thiazolidinyl, isothiazolyl, quinuclidinyl, isothiazolidinyl, isoindolyl, indolinyl, isoindolinyl, octahydroindolyl, octahydroisoindolyl, decahydroisoquinolyl, benzimidazolyl, thiadiazolyl, benzopyranyl, tetrahydrofuryl, tetrahydropyranyl, thienyl, benzothienyl, thiamorpholinyl, thiamorpholinyl sulfoxide, thiamorpholinyl sulfone, dioxaphospholanyl, oxadiazolyl, chromanyl, and isochromanyl. The heteroaryl ring radical may be attached to the main structure at any heteroatom or carbon atom. The term “substituted heteroaryl” also includes ring systems substituted with one or more oxide (═O) substituents, such as pyridinyl N-oxides.

The term “arylalkyl”, unless otherwise specified, refers to an aryl group as defined above directly bonded to an alkyl group as defined above, e.g., —CH₂C₆H₅ and —C₂H₅C₆H₅.

In the present disclosure, the term “optionally substituted aryl” as used herein by itself or as part of another group means that the aryl as defined above is either unsubstituted or substituted with one to five substituents independently selected from the group consisting of halogen, hydroxy, nitro, cyano, —SCH₃, —SCF₃, —NR₁₀R₁₁, —C(═O)NR₁₀R₁₁, —C(═O)R₁₃, C₁₋₆ alkyl, C₂₋₆ alkenyl, C₂₋₆ alkynyl, C₁₋₆ alkoxy, C₁₋₆ haloalkyl, haloalkoxy, optionally substituted C₃₋₁₂ cycloalkyl, optionally substituted C₆-C₁₄ aryl, optionally substituted 5- to 14-membered heteroaryl, and optionally substituted 3- to 14-membered heterocyclic ring, wherein R₁₀ and R₁₁ are independently selected from the group consisting of hydrogen and C₁₋₆ alkyl; or R₁₀ and R₁₁ taken together with the nitrogen atom to which they are attached form a 3- to 12-membered heterocyclic ring and R₁₃ is C1-4 alkyl.

In one embodiment, the optionally substituted aryl is an optionally substituted phenyl. In one embodiment, the optionally substituted phenyl has four substituents. In another embodiment, the optionally substituted phenyl has three substituents. In another embodiment, the optionally substituted phenyl has two substituents. In another embodiment, the optionally substituted phenyl has one substituent. Non-limiting exemplary substituted aryl groups include 2-methylphenyl, 2-methoxyphenyl, 2-fluorophenyl, 2-chlorophenyl, 2-bromophenyl, 3-methylphenyl, 3-methoxyphenyl, 3-fluorophenyl, 3-chlorophenyl, 4-methylphenyl, 4-ethylphenyl, 4-methoxyphenyl, 4-fluorophenyl, 4-chlorophenyl, 4-bromophenyl, 4-triflurophenyl, 2,6-di-fluorophenyl, 2,6-di-chlorophenyl, 2-methyl, 3-methoxyphenyl, 2-ethyl, 3-methoxyphenyl, 3,4-di-methoxyphenyl, 3,5-di-fluorophenyl, 3,4-di-chlorophenyl, 3,5-di-methylphenyl, 3,5-dimethoxy, 4-methylphenyl, 2-fluoro-3-chlorophenyl, 3-chloro-4-fluorophenyl.

Additional non-limiting exemplary substituted aryl groups include 4-isopropylphenyl, 4-chlorophenyl, 4-bromophenyl, 4-fluorophenyl, 4-methoxyphenyl, 4-methylphenyl, and 4-trifluoromethylphenyl.

The term optionally substituted aryl is also meant to include groups having fused optionally substituted cycloalkyl and fused optionally substituted heterocyclic rings. Non-limiting examples include:

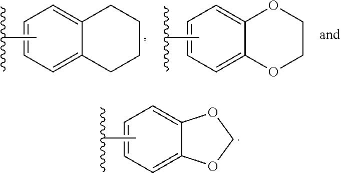

Certain of the compounds described herein may contain one or more asymmetric centers and can thus give rise to enantiomers, diastereomers, and other stereoisomeric forms that can be defined, in terms of absolute stereochemistry, as (R)— or (S)—. The present chemical entities, pharmaceutical compositions and methods are meant to include all such possible isomers, including racemic mixtures, optically pure forms and intermediate mixtures. Non-limiting examples of intermediate mixtures include a mixture of isomers in a ratio of 10:90, 13:87, 17:83, 20:80, or 22:78. Optically active (R)- and (S)-isomers can be prepared using chiral synthons or chiral reagents or resolved using conventional techniques. When the compounds described herein contain olefinic double bonds or other centers of geometric asymmetry, and unless specified otherwise, it is intended that the compounds include both E and Z geometric isomers.

The terms “tautomer” and “tautomers” refer to compounds which are characterized by relatively easy interconversion of isomeric forms in equilibrium. These isomers are intended to be covered by this invention. “Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g. in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

Additionally, the present invention also includes the compounds which differ only in the presence of one or more isotopically enriched atoms for example replacement of hydrogen with deuterium or tritium, or the replacement of a carbon by ¹³C- or ¹⁴C-enriched carbon.

The compounds of the present invention may also contain unnatural proportions of atomic isotopes at one or more of atoms that constitute such compounds. For example, the compounds may be radio-labelled with radioactive isotopes, such as for example tritium (³H), iodine-125 (¹²⁵I) or carbon-14 (¹⁴C). All isotopic variations of the compounds of the present invention, whether radioactive or not, are encompassed within the scope of the present invention.

The term “comprising” (and related terms such as “comprise,” “comprises,” “having” or “including”) includes those embodiments, for example, an embodiment of any composition of matter, composition, method, or process, or the like, that “consist of” or “consist essentially of” the described features.

The term “subject” or “patient” refers to an animal, such as a mammal, for example a human. The methods and uses described herein can be useful in both human therapeutics and veterinary applications (e.g., dogs, cats, cows, sheep, pigs, horses, goats, chickens, turkeys, ducks, and geese).

In some embodiments, the subject is a mammal, and in some embodiments, the subject is a human.

The term “pharmaceutically acceptable excipient” includes, but is not limited to, any and all solvents, dispersion media, coatings, antibacterial and antifungal agents, isotonic and absorption delaying agents, one or more suitable diluents, fillers, salts, disintegrants, binders, lubricants, glidants, wetting agents, controlled release matrices, colorants/flavoring, carriers, buffers, stabilizers, solubilizers, and combinations thereof. Except insofar as any conventional media or agent is incompatible with the active ingredient, its use in the therapeutic compositions of the invention is contemplated. Supplementary active ingredients can also be incorporated into the compositions.

Dxr inhibitors are described in, for example, U.S. Pat. No. 9,593,136 and International Publication Nos. WO 19/005982 and WO 17/12780, each of which are incorporated herein by reference in their entirety.

The present invention provides a series of compounds as antimicrobial agents that work via DXR inhibition.

In one aspect, the present invention relates to a compound of formula (I)

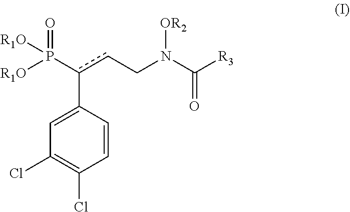

or a tautomer thereof, stereoisomer thereof, prodrug thereof, or pharmaceutically acceptable salt thereof,
wherein
---- represents a bond or is absent;
each R₁ is, independently, —NH₄, —N(alkyl)₄, —N(aryl)₄, C₁₋₄ alkyl (e.g., —CH₂CH₃), or —(CR^{a}R^{b})ₘ—O(C═O)—C₁₋₆ alkyl (e.g., —CH₂—O—C(═O)—C(CH₃)₃), wherein the atom at the left is attached to the oxygen atom;
R₂ is H, —(CR^{c}R^{d})ₙ-aryl, or —(CR^{c}R^{d})ₙ-heteroaryl, wherein the atom at the left is attached to the oxygen atom;
R₃ is H, C₁₋₆ alkyl (e.g., methyl), C₁₋₆ haloalkyl, C₃₋₆ cycloalkyl, C₁₋₆ alkoxy, C₁₋₆ haloalkoxy, C₃₋₆ cycloalkoxy, —(CR_{c}R_{d})ₚ-aryl or or —(CR^{c}R^{d})ₙ-heteroaryl;
each of R^{a}, R^{b}, R^{c}, and R^{d} is independently H, halogen, or C₁₋₄ alkyl (e.g. methyl, or ethyl);
each of m and n is independently 1, 2, 3, or 4; and
p is 0, 1, 2, 3, or 4.
wherein each aryl or heteroaryl is, independently, optionally substituted with up to five R⁴ selected from the group consisting of halogen, hydroxyl, cyano, amino, (C₁₋₆ alkyl)amino, di(C₁₋₆ alkyl)amino, C₁₋₆ alkyl, C₁₋₆ haloalkyl, C₃₋₆ cycloalkyl, C₁₋₆ alkoxy, C₁₋₆ haloalkoxy, C₃₋₆ cycloalkoxy, arylalkyl (e.g., benzyl) and heteroaryl (e.g., pyridine, furan, thiophene, indole);
with the proviso that when ---- is absent, each R₁ is ethyl or —CH₂—O—C(═O)—C(CH₃)₃ and R₃ is methyl, then R₂ is not

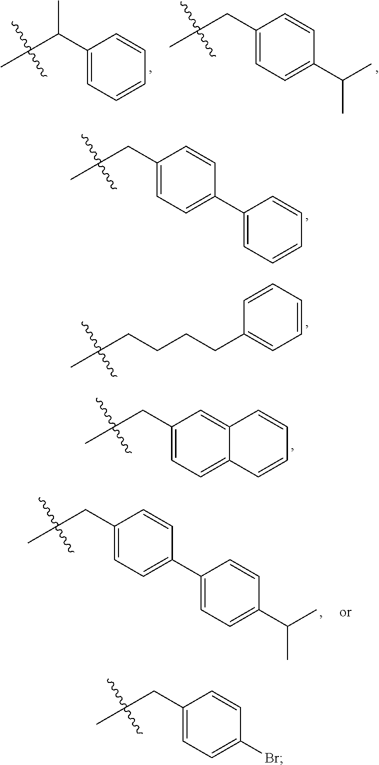

where the squiggly line ( [symbol] ) represents the point of attachment of R₂ to the reset of the molecule.

In another aspect, the present invention relates to a compound of formula (I)

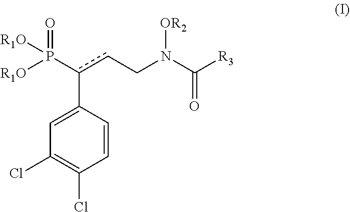

or a tautomer thereof, stereoisomer thereof, prodrug thereof, or pharmaceutically acceptable salt thereof,
wherein
---- represents a bond or is absent;
each R₁ is, independently, —NH₄, —N(alkyl)₄, —N(aryl)₄, C₁₋₄ alkyl (e.g., —CH₂CH₃), or —(CR^{a}R^{b})ₘ—O(C═O)—C₁₋₆ alkyl (e.g., —CH₂—O—C(═O)—C(CH₃)₃), wherein the atom at the left is attached to the oxygen atom;
R₂ is H, —(CR^{c}R^{d})ₙ-aryl, or —(CR^{c}R^{d})ₙ-heteroaryl, wherein the atom at the left is attached to the oxygen atom;
R₃ is H, C₁₋₆ alkyl (e.g., methyl), C₁₋₆ haloalkyl, C₃₋₆ cycloalkyl, C₁₋₆ alkoxy, C₁₋₆ haloalkoxy, C₃₋₆ cycloalkoxy, —(CR_{c}R_{d})ₚ-aryl or or —(CR^{c}R^{d})ₙ-heteroaryl;
each of R^{a}, R^{b}, R^{c}, and R^{d} is independently H, halogen, or C₁₋₄ alkyl (e.g. methyl, or ethyl);
each of m and n is independently 1, 2, 3, or 4; and
p is 0, 1, 2, 3, or 4;
wherein each aryl or heteroaryl is, independently, optionally substituted with up to five R⁴ selected from the group consisting of halogen, hydroxyl, cyano, amino, (C₁₋₆ alkyl)amino, di(C₁₋₆ alkyl)amino, C₁₋₆ alkyl, C₁₋₆ haloalkyl, C₃₋₆ cycloalkyl, C₁₋₆ alkoxy, C₁₋₆ haloalkoxy, C₃₋₆ cycloalkoxy, arylalkyl (e.g., benzyl) and heteroaryl (e.g., pyridine, furan, thiophene, indole);
with the proviso that when ---- is absent, each R₁ is ethyl or —CH₂—O—C(═O)—C(CH₃)₃ and R₂ is

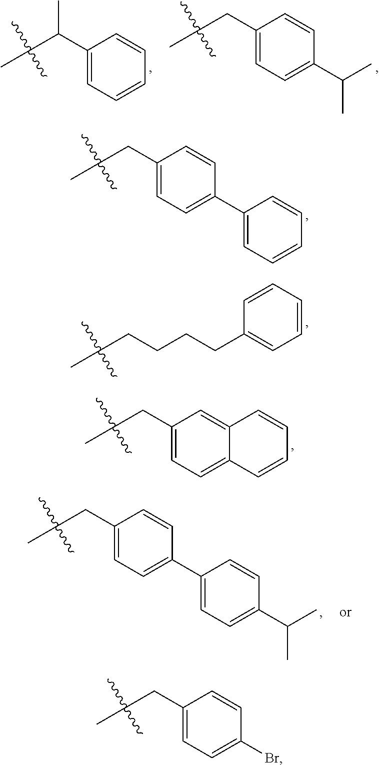

where the squiggly line ( [symbol] ) represents the point of attachment of R₂ to the reset of the molecule,
then R₃ is not methyl.
In a further embodiment of this specific aspect of the invention, when ----, R₁ and R₂ are as described in the proviso above, then R₃ is not methyl or ethyl. In yet a further embodiment of this specific aspect of the invention, when ----, R₁ and R₂ are as described in the proviso above, then R₃ is not C₁₋₆ alkyl.

In one embodiment, the compound of formula (I) is not

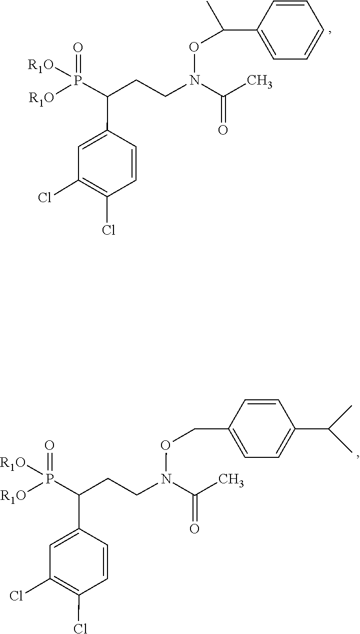

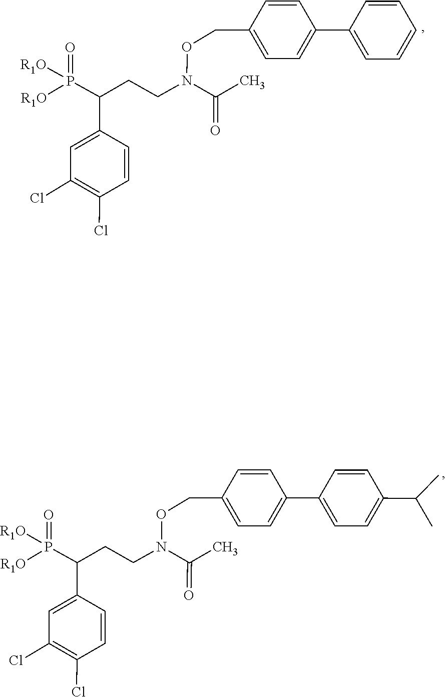

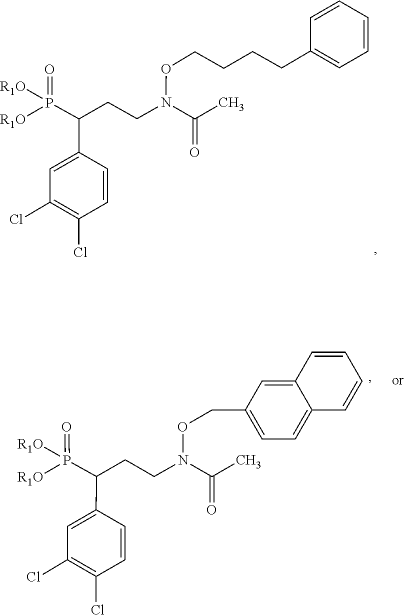

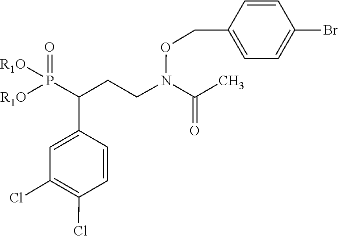

wherein each R₁ is ethyl or each R₁ is —CH₂—O(C═O)—C(CH₃)₃ (pivaloyloxymethyl, POM).

In another aspect, the present invention relates to a compound of formula (I)

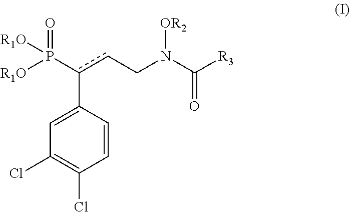

or a tautomer thereof, stereoisomer thereof, prodrug thereof, or pharmaceutically acceptable salt thereof,
wherein
---- represents a bond or is absent;
each R₁ is, independently, —NH₄, —N(alkyl)₄, —N(aryl)₄, C₁₋₄ alkyl (e.g., —CH₂CH₃), or —(CR^{a}R^{b})ₘ—O(C═O)—C₁₋₆ alkyl (e.g., —CH₂—O—C(═O)—C(CH₃)₃), wherein the atom at the left is attached to the oxygen atom;
R₂ is H, —(CR^{c}R^{d})ₙ-aryl, or —(CR^{c}R^{d})ₙ-heteroaryl, wherein the atom at the left is attached to the oxygen atom;
R₃ is CH₃;
each of R^{a}, R^{b}, R^{c}, and R^{d} is independently H, halogen, or C₁₋₄ alkyl (e.g. methyl, or ethyl);
each of m and n is independently 1, 2, 3, or 4; and
p is 0, 1, 2, 3, or 4;
wherein each aryl or heteroaryl is, independently, optionally substituted with up to five R⁴ selected from the group consisting of halogen, hydroxyl, cyano, amino, (C₁₋₆ alkyl)amino, di(C₁₋₆ alkyl)amino, C₁₋₆ alkyl, C₁₋₆ haloalkyl, C₃₋₆ cycloalkyl, C₁₋₆ alkoxy, C₁₋₆ haloalkoxy, C₃₋₆ cycloalkoxy, arylalkyl (e.g., benzyl) and heteroaryl (e.g., pyridine, furan, thiophene, indole);
   with the proviso that the compound of formula (I) is not

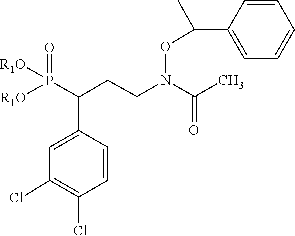

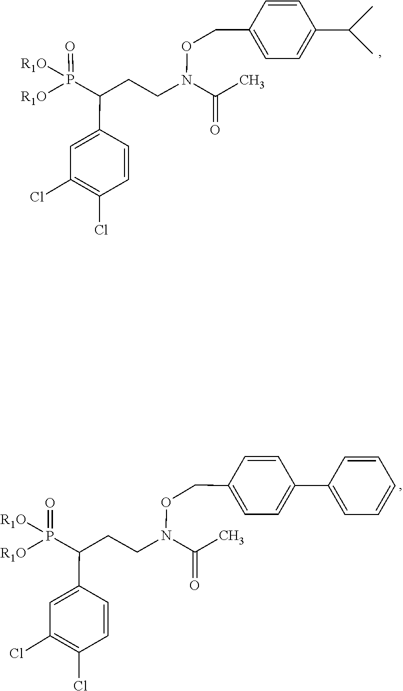

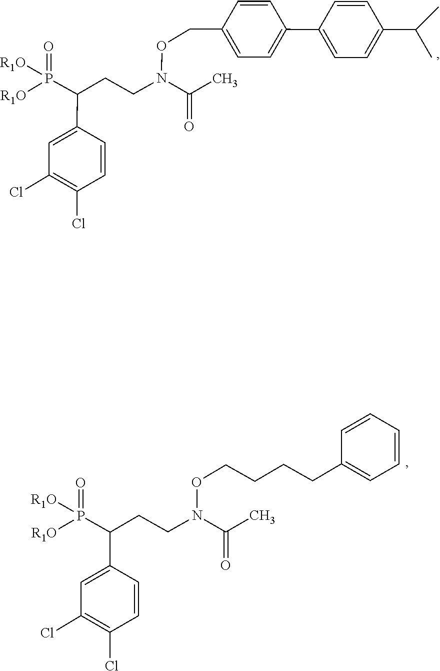

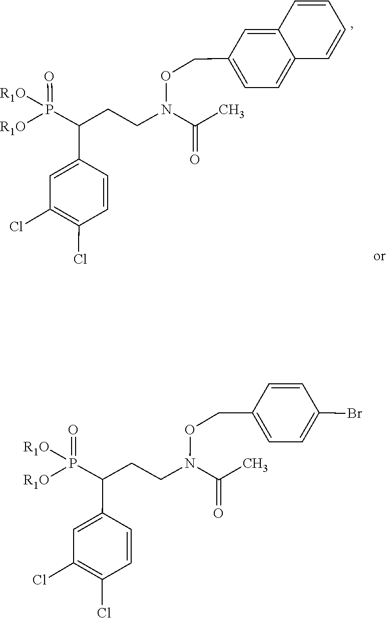

wherein each R₁ is ethyl or each R₁ is —CH₂—O(C═O)—C(CH₃)₃ (pivaloyloxymethyl, POM).

In some embodiments of any of the compounds of formula (I), the compound is a mono-salt. In some embodiments of any of the compounds of formula (I), the compound is a di-salt.

In some embodiments of any of the compounds of formula (I), the salt is a quaternary ammonium salt. In some of any of the compounds of formula (I), the salt is a NH₄ salt. In some embodiments of any of the compounds of formula (I), the salt is a di-quaternary ammonium salt. In some embodiments of any of the compounds of formula (I), the salt is a di-NH₄ salt.

In some embodiments of any of the compounds of formula (I), each R₁ is NH₄.

In some embodiments of any of the compounds of formula (I), each R₁ is C₁₋₄ alkyl.

In some embodiments of any of the compounds of formula (I), each R₁ is ethyl.

In some embodiments of any of the compounds of formula (I), each R₁ is —(CR^{a}R^{b})m-O(C═O)—C₁₋₆ alkyl. In some embodiments of any of the compounds of formula (I), each R₁ is —CH₂O(C═O)—C₁₋₆ alkyl. In some embodiments of any of the compounds of formula (I), each R₁ is —CH(CH3)—O(C═O)—C₁₋₆ alkyl. In some embodiments of any of the compounds of formula (I), each R₁ is —CH₂—O(C═O)—C(CH₃)₃ (pivaloyloxymethyl, POM).

In some embodiments of any of the compounds of formula (I), R₂ is —(CR^{c}R⁴)ₙ-aryl, wherein aryl is an optionally substituted phenyl, biphenyl, or naphthyl. In some embodiments of any of the compounds of formula (I), R₂ is (CH₂)ₙ-aryl. In some embodiments, R₂ is CH₂-aryl. In some embodiments of any of the compounds of formula (I), R₂ is CH(CH₃)-aryl.

In some embodiments of any of the compounds of formula (I), the aryl group in R₂ is phenyl optionally substituted with up to five R₄.

In some embodiments of any of the compounds of formula (I), R₄ is selected from the group consisting of halogen, hydroxyl, cyano, amino, (C₁₋₆ alkyl)amino, di(C₁₋₆ alkyl)amino, C₁₋₆ alkyl, C₁₋₆ haloalkyl, C₃₋₆ cycloalkyl, C₁₋₆ alkoxy, C₁₋₆ haloalkoxy, and C₃₋₆ cycloalkoxy. In some embodiments, R₄ is halogen, cyano, CF₃, methyl, ethyl, n-propyl, isopropyl, methoxy, ethoxy, n-propoxyl, or isopropoxyl.

In some embodiments of any of the compounds of formula (I), the aryl group in R₂ is phenyl without a substituent. In some embodiments of any of the compounds of formula (I), the aryl group in R₂ is phenyl substituted with one R₄ at the para-position.

In some embodiments of any of the compounds of formula (I), the aryl group in R₂ is naphthyl optionally substituted with up to five R₄. In some embodiments of any of the compounds of formula (I), the aryl group in R₂ is 1-naphthyl. In some embodiments of any of the compounds of formula (I), the aryl group in R₂ is 2-naphthyl.

In some embodiments of any of the compounds of formula (I), R₂ is CH(CH₃)Ph, CH₂(4-biphenyl), CH₂(2-napthyl), CH₂(4-iPr4-biphenyl), CH₂CH₂CH₂CH₂phenyl, CH(CH₃)(4-chlorophenyl), CH(CH₃)(4-bromophenyl), CH(CH₃)(4-flurophenyl), CH(CH₃)(4-methoxyphenyl), CH(CH₃)(4-trifluromethylphenyl) or CH(CH₃)(4-methylphenyl).

In some embodiments of any of the compounds of formula (I), n is 1, 2, or 3. In some embodiments of any of the compounds of formula (I), n is 1 or 2. In some embodiments of any of the compounds of formula (I), n is 1.

In some embodiments of any of the compounds of formula (I), R₃ is H, C₁₋₃ alkyl, C₁₋₃ haloalkyl, C₁₋₃ alkoxy, or C₁₋₃ haloalkoxy

In some embodiments of any of the compounds of formula (I), R₃ is H.

In some embodiments of any of the compounds of formula (I), R₃ is C₁₋₃ alkyl. In some embodiments of any of the compounds of formula (I), R₃ is —CH₃.

In some embodiments of any of the compounds of formula (I), R₃ is C₁₋₃ haloalkyl. In some embodiments of any of the compounds of formula (I), R₃ is —OCH₃. In some embodiments of any of the compounds of formula (I), R₃ is C₁₋₃ haloalkyl. In some embodiments of any of the compounds of formula (I), R₃ is —CF₃. In some embodiments of any of the compounds of formula (I), R₃ is H, —CH₃, —CF₃, or —OCH₃.

In some embodiments of any of the compounds of formula (I), R₃ is (CH₂)p-aryl, wherein p is 0, 1, 2, or 3. In some embodiments of any of the compounds of formula (I), R₃ is phenyl. In some embodiments of any of the compounds of formula (I), R₃ is benzyl.

In some embodiments of any of the compounds of formula (I), the compound of formula (I) has the structure of formula IA:

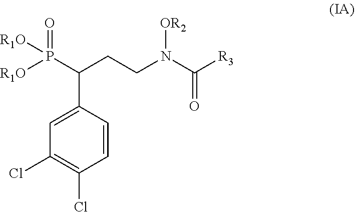

wherein R₁, R₂ and R₃ are as described in any embodiment herein.

In some embodiments of any of the compounds of formula (I), the compound of formula (I) has the structure of formula IB:

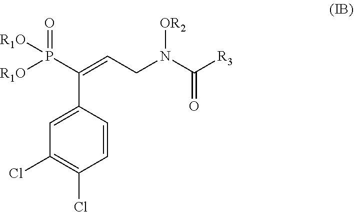

wherein R₁, R₂ and R₃ are as described in any embodiment herein.

Examples of compounds of the present invention include, but are not limited to, those shown in Tables 1 and 2 below, and tautomers, stereoisomers, prodrugs and pharmaceutically acceptable salt thereof.

TABLE 1

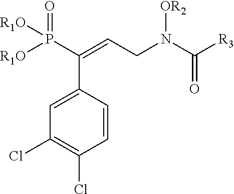

| Compound # | R¹ | R² | R³ |
|---|---|---|---|
| 1 | POM | CH(CH₃)Ph | CH₃ |
| 2 | POM | CH₂(4-biphenyl) | CH₃ |
| 3 | POM | CH₂(4-iPrPh) | CH₃ |
| 4 | POM | CH₂(2-napthyl) | CH₃ |
| 5 | POM | CH₂(4-iPr4-biphenyl) | CH₃ |
| 6 | POM | CH₂CH₂CH₂CH₂phenyl | CH₃ |
| 7 | POM | CH(CH₃)(4-chlorophenyl) | CH₃ |
| 8 | POM | CH(CH₃)(4-bromophenyl) | CH₃ |
| 9 | POM | CH(CH₃)(4-flurophenyl) | CH₃ |
| 10 | POM | CH(CH₃)(4-methoxyphenyl) | CH₃ |
| 11 | POM | CH(CH₃)(4-trifluromethylphenyl) | CH₃ |
| 12 | POM | CH(CH₃)(4-methylphenyl) | CH₃ |
| 13 | NH₄ | CH(CH₃)Ph | CH₃ |
| 14 | NH₄ | CH₂(4-biphenyl) | CH₃ |
| 15 | NH₄ | CH₂(4-iPrPh) | CH₃ |
| 16 | NH₄ | CH₂(2-napthyl) | CH₃ |
| 17 | NH₄ | CH₂(4-iPr-4-biphenyl) | CH₃ |
| 18 | NH₄ | CH₂CH₂CH₂CH₂phenyl | CH₃ |
| 19 | NH₄ | CH(CH₃)(4-chlorophenyl) | CH₃ |
| 20 | NH₄ | CH(CH₃)(4-bromophenyl) | CH₃ |
| 21 | NH₄ | CH(CH₃)(4-flurophenyl) | CH₃ |
| 22 | NH₄ | CH(CH₃)(4-methoxyphenyl) | CH₃ |
| 23 | NH₄ | CH(CH₃)(4-trifluromethylphenyl) | CH₃ |
| 24 | NH₄ | CH(CH₃)(4-methylphenyl) | CH₃ |
| 25 | Et | CH(CH₃)Ph | CH₃ |
| 26 | Et | CH₂(4-biphenyl) | CH₃ |
| 27 | Et | CH₂(4-iPrPh) | CH₃ |
| 28 | Et | CH₂(2-napthyl) | CH₃ |
| 29 | Et | CH₂(4-iPr4-biphenyl) | CH₃ |
| 30 | Et | CH₂CH₂CH₂CH₂phenyl | CH₃ |
| 31 | Et | CH(CH₃)(4-chlorophenyl) | CH₃ |
| 32 | Et | CH(CH₃)(4-bromophenyl) | CH₃ |
| 33 | Et | CH(CH₃)(4-flurophenyl) | CH₃ |
| 34 | Et | CH(CH₃)(4-methoxyphenyl) | CH₃ |
| 35 | Et | CH(CH₃)(4-trifluromethylphenyl) | CH₃ |
| 36 | Et | CH(CH₃)(4-methylphenyl) | CH₃ |

TABLE 2

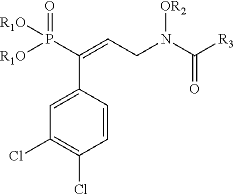

| Compound # | R¹ | R² | R³ |
|---|---|---|---|
| 37 | POM | CH(CH₃)Ph | CH₃ |
| 38 | POM | CH₂(4-biphenyl) | CH₃ |
| 39 | POM | CH₂(4-iPrPh) | CH₃ |
| 40 | POM | CH₂(2-napthyl) | CH₃ |
| 41 | POM | CH₂(4-iPr4-biphenyl) | CH₃ |
| 42 | POM | CH₂CH₂CH₂CH₂phenyl | CH₃ |
| 43 | POM | CH(CH₃)(4-chlorophenyl) | CH₃ |
| 44 | POM | CH(CH₃)(4-bromophenyl) | CH₃ |
| 45 | POM | CH(CH₃)(4-flurophenyl) | CH₃ |
| 46 | POM | CH(CH₃)(4-methoxyphenyl) | CH₃ |
| 47 | POM | CH(CH₃)(4-trifluromethylphenyl) | CH₃ |
| 48 | POM | CH(CH₃)(4-methylphenyl) | CH₃ |
| 49 | NH₄ | CH(CH₃)Ph | CH₃ |
| 50 | NH₄ | CH₂(4-biphenyl) | CH₃ |
| 51 | NH₄ | CH₂(4-iPrPh) | CH₃ |
| 52 | NH₄ | CH₂(2-napthyl) | CH₃ |
| 53 | NH₄ | CH₂(4-iPr-4-biphenyl) | CH₃ |
| 54 | NH₄ | CH₂CH₂CH₂CH₂phenyl | CH₃ |
| 55 | NH₄ | CH(CH₃)(4-chlorophenyl) | CH₃ |
| 56 | NH₄ | CH(CH₃)(4-bromophenyl) | CH₃ |
| 57 | NH₄ | CH(CH₃)(4-flurophenyl) | CH₃ |
| 58 | NH₄ | CH(CH₃)(4-methoxyphenyl) | CH₃ |
| 59 | NH₄ | CH(CH₃)(4-trifluromethylphenyl) | CH₃ |
| 60 | NH₄ | CH(CH₃)(4-methylphenyl) | CH₃ |
| 61 | Et | CH(CH₃)Ph | CH₃ |
| 62 | Et | CH₂(4-biphenyl) | CH₃ |
| 63 | Et | CH₂(4-iPrPh) | CH₃ |
| 64 | Et | CH₂(₂-napthyl) | CH₃ |
| 65 | Et | CH₂(4-iPr4-biphenyl) | CH₃ |
| 66 | Et | CH₂CH₂CH₂CH₂phenyl | CH₃ |
| 67 | Et | CH(CH₃)(4-chlorophenyl) | CH₃ |
| 68 | Et | CH(CH₃)(4-bromophenyl) | CH₃ |
| 69 | Et | CH(CH₃)(4-flurophenyl) | CH₃ |
| 70 | Et | CH(CH₃)(4-methoxyphenyl) | CH₃ |
| 71 | Et | CH(CH₃)(4-trifluromethylphenyl) | CH₃ |
| 72 | Et | CH(CH₃)(4-methylphenyl) | CH₃ |
| A | NH₄ | H | CH₃ |
| B | NH₄ | CH₂(2-napthyl) | CH₃ |

### Pharmaceutical Compositions

The present invention provides a pharmaceutical composition comprising one or more compounds of the present invention, or a pharmaceutically acceptable salt thereof. The pharmaceutical compositions described herein may include one or more additional active ingredients as described herein. The pharmaceutical composition may be administered for any of the disorders described herein.

The pharmaceutical compositions described herein are typically formulated to provide a therapeutically effective amount of a compound of the present invention as the active ingredient. Where desired, the pharmaceutical compositions contain a compound of the present invention as the active ingredient and one or more pharmaceutically acceptable carriers or excipients, such as inert solid diluents and fillers, diluents, including sterile aqueous solution and various organic solvents, permeation enhancers, solubilizers and adjuvants.

The pharmaceutical compositions can be administered alone or in combination with one or more other agents, which are also typically administered in the form of pharmaceutical compositions. Where desired, the subject compounds and other agent(s) may be mixed into a preparation or both components may be formulated into separate preparations to use them in combination separately or at the same time.

Methods and uses described herein include administration of a compound of the present invention by itself, or in combination as described herein, and in each case optionally including one or more suitable diluents, fillers, salts, disintegrants, binders, lubricants, glidants, wetting agents, controlled release matrices, colorants/flavorings, carriers, excipients, buffers, stabilizers, solubilizers, and combinations thereof.

Preparations of various pharmaceutical compositions are well known in the art., see, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2003; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999), all of which are incorporated by reference herein in their entirety.

The compounds or pharmaceutical compositions of the present invention can be administered by any route that enables delivery of the compound(s) to their intended site of action, such as oral routes, intraduodenal routes, parenteral injection (including intravenous, intraarterial, subcutaneous, intramuscular, intravascular, intraperitoneal or infusion), topical administration (e.g. transdermal application), rectal administration, via local delivery by catheter or stent or through inhalation. The compounds can also be administered intraadiposally or intrathecally.

The compositions described herein can be administered in solid, semi-solid, liquid or gaseous form, or may be in dried powder, such as lyophilized form. The pharmaceutical compositions can be packaged in forms convenient for delivery, including, for example, solid dosage forms such as capsules, sachets, cachets, gelatins, papers, tablets, capsules, suppositories, pellets, pills, troches, and lozenges. The type of packaging will generally depend on the desired route of administration. Implantable sustained release formulations are also contemplated, as are transdermal formulations.

### Synthetis

The following general methodology described in the schemes below provides the manner and process of making and using the compounds of the present invention and are illustrative rather than limiting. Further modification of the provided methodology may also be devised to achieve and serve the purpose of the invention. Accordingly, there may be other embodiments which fall within the spirit and scope of the invention as defined by this specification. In schemes 1 and 2, substituent R shown is the same as substituent R₂ in the compounds of formula (I) described herein.

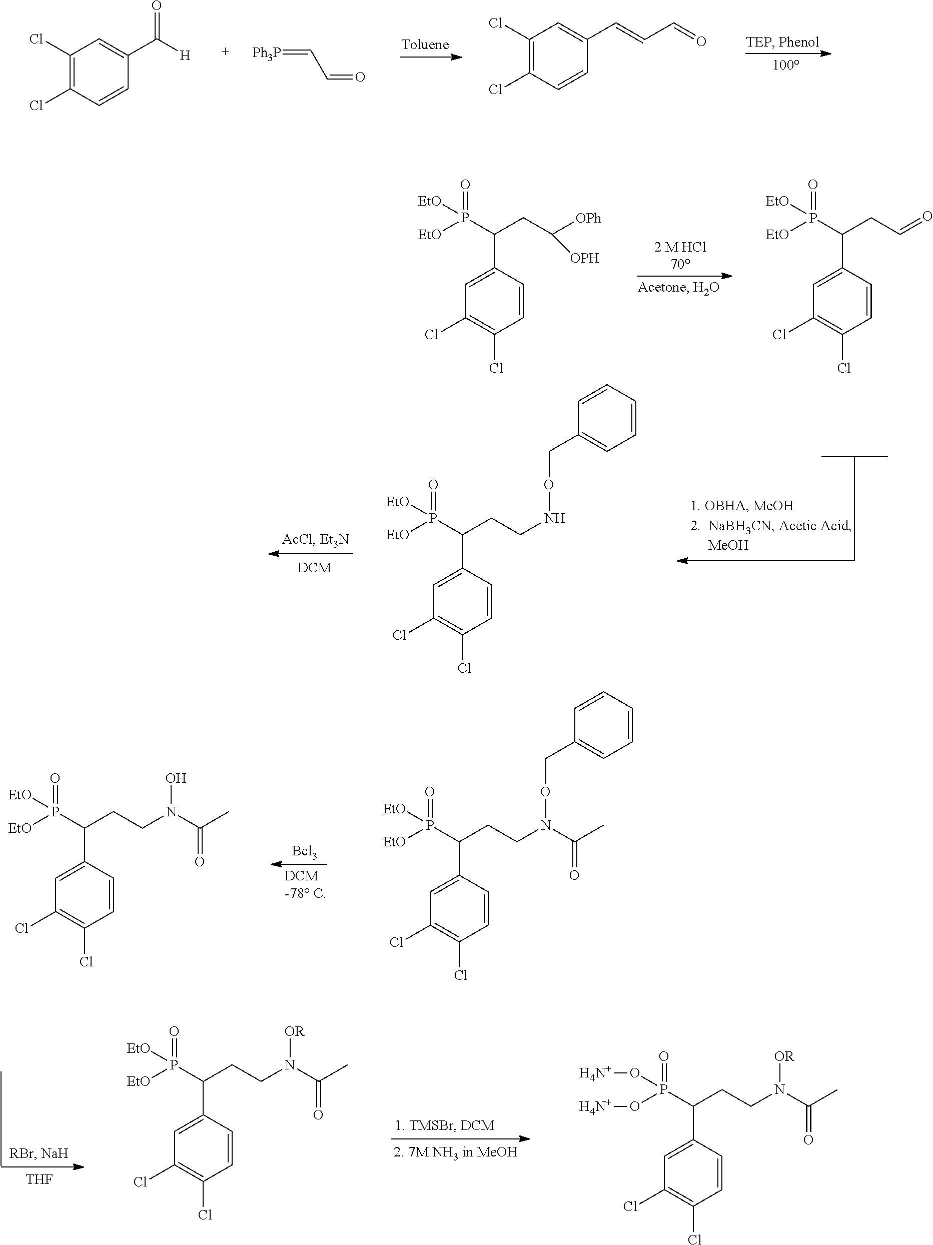

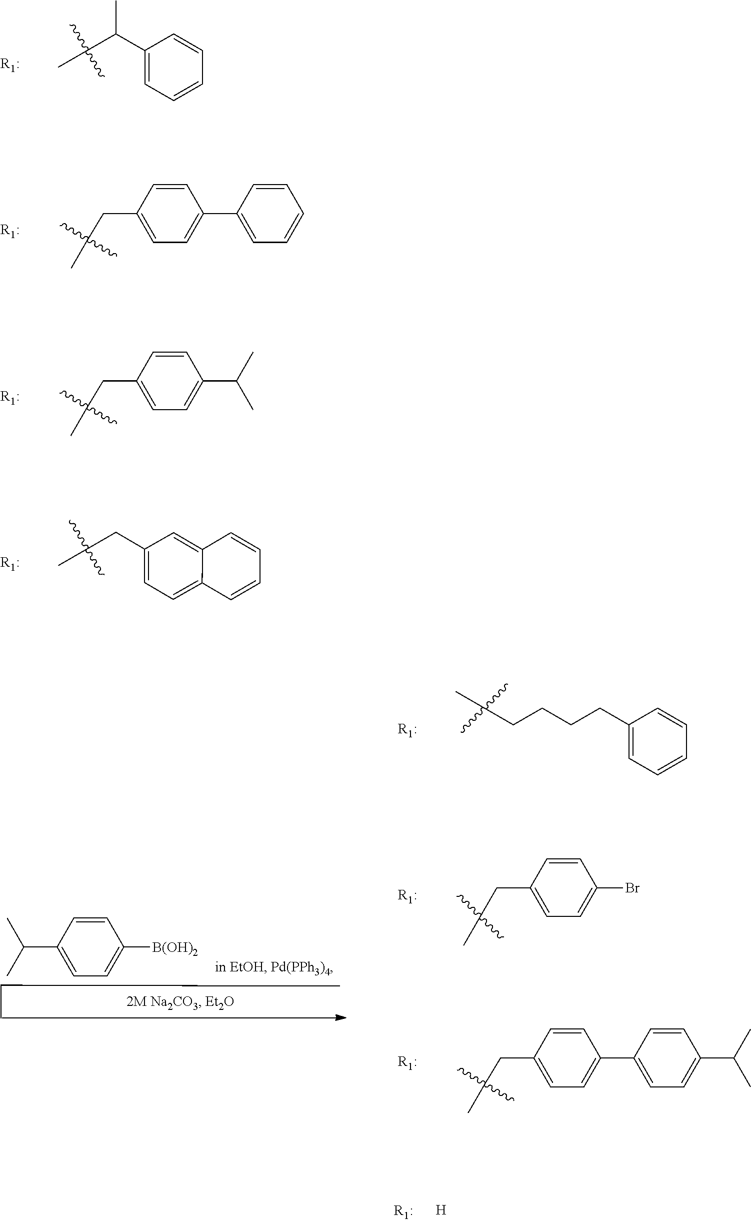

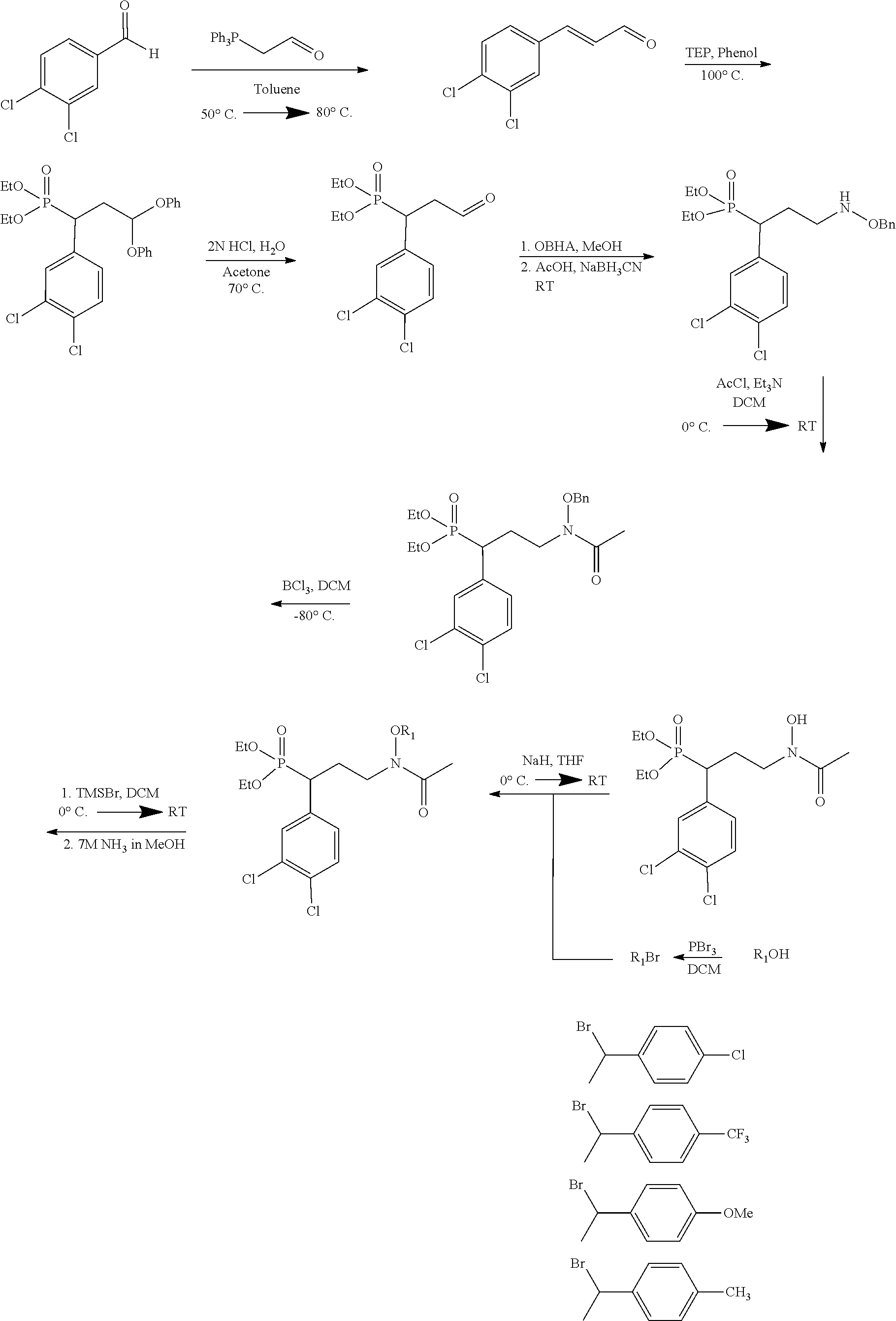

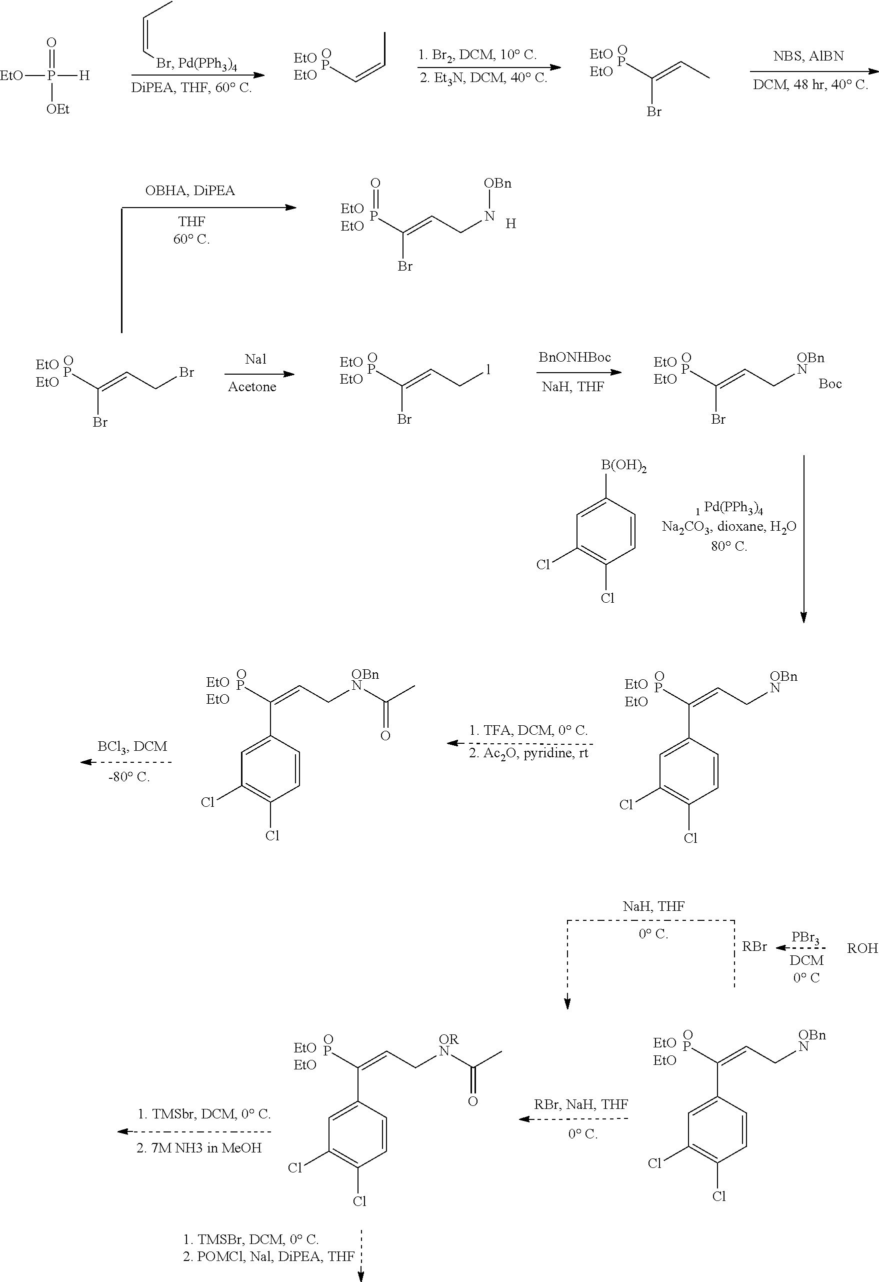

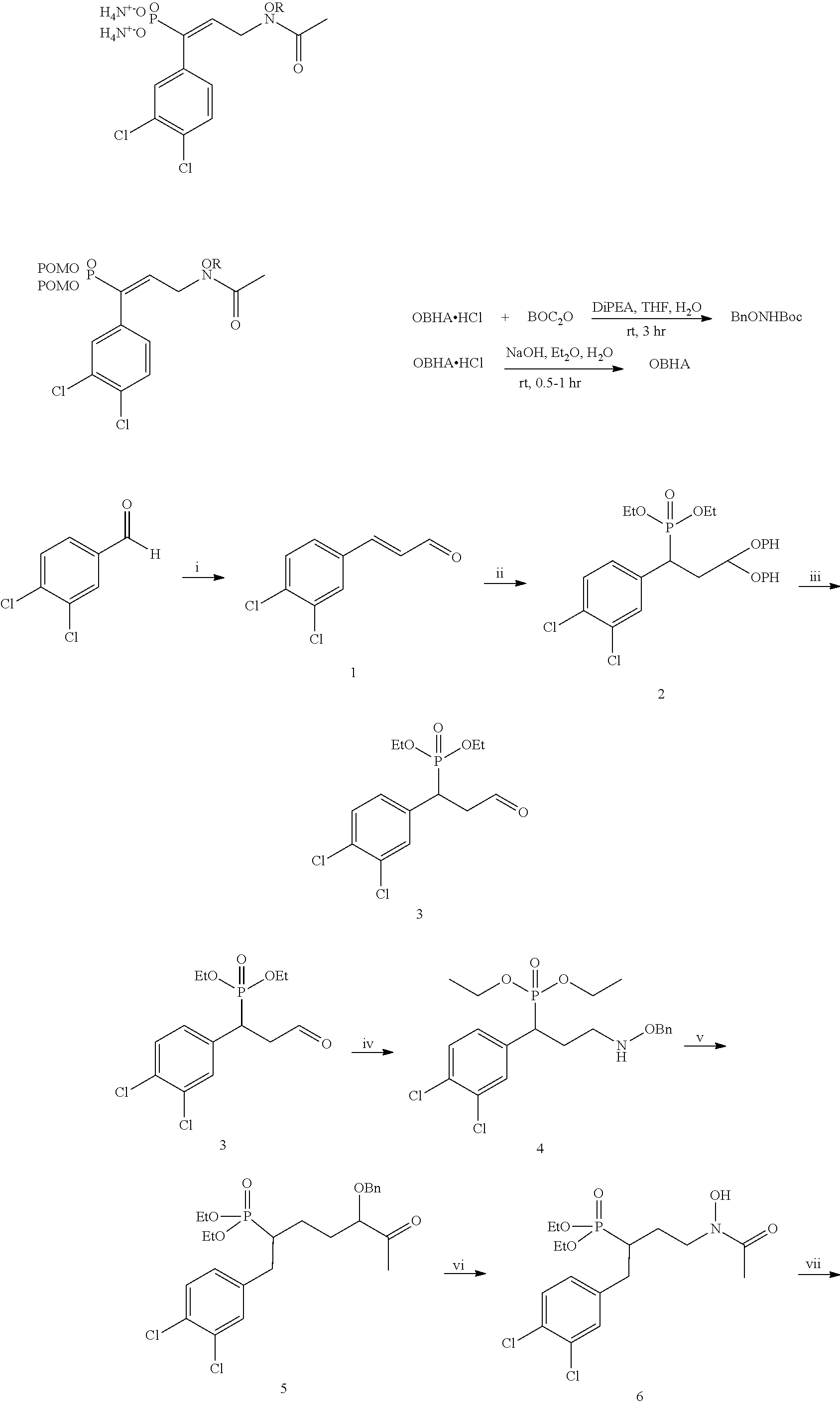

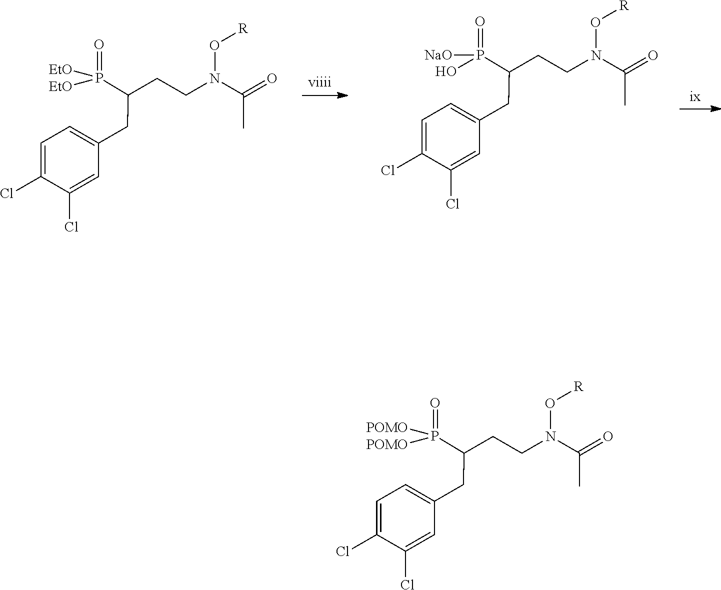

### Mycobacterium tuberculosis Inhibition Data

Exemplary Mycobacterium tuberculosis minimum inhibitory concentration (MIC) data (μg/ml) for compounds of the present invention is provided in Table 3 below.

TABLE 3

| | | | | | 1-week MIC | 2-week MIC |
|---|---|---|---|---|---|---|
| | 1-week MIC | 2-week MIC | 1-week MIC | 2-week MIC | 7H9/glycerol/ | 7H9/glycerol/ |
| | 7H9/glucose/ | 7H9/glucose/ | 7H9/glucose/ | 7H9/glucose/ | glucose/ | glucose/ |
| | casitone/Tx | casitone/Tx | BSA/Tx | BSA/Tx | BSA/Tween | BSA/Tween |
| [symbol] | >100 | >100 | >100 | >100 | >100 | >100 |
| 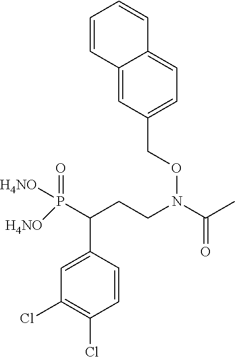 | 12.5 | 25 | >100 | >100 | >100 | >100 |
| 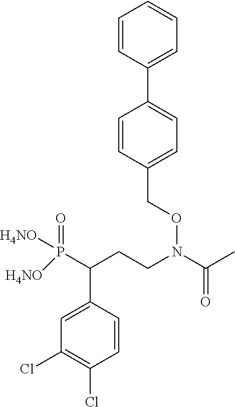 | 12.5 | 25 | >100 | >100 | >100 | >100 |
| 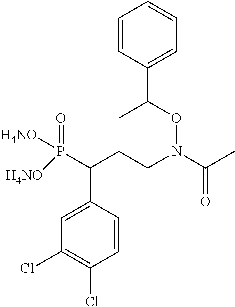 | >100 | >100 | >100 | >100 | >100 | >100 |
| 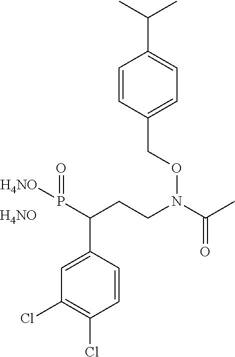 | 6.25 | 25 | >100 | >100 | >=100 | >=100 |
| 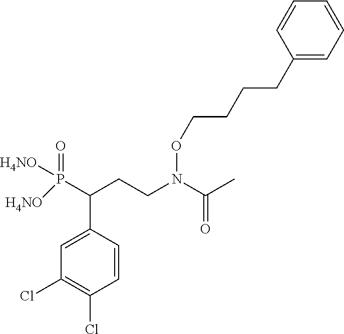 | 1.56 | 6.25 | >100 | >100 | >100 | >100 |
| Isoniazid | 0.02 | 0.02 | 0.02 | 0.03 | 0.03 | 0.039 |

[symbol]

indicates data missing or illegible when filed

### Plasmodium falciparum Inhibition Data

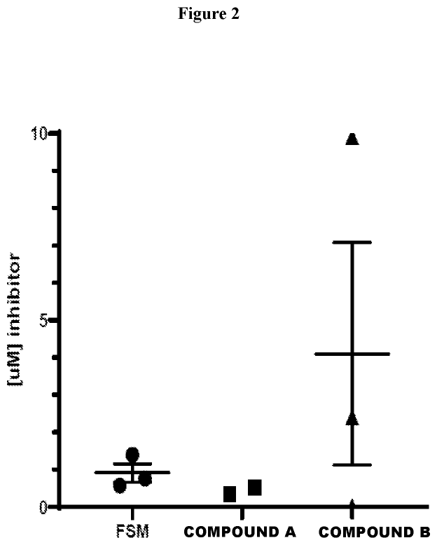

Exemplary Plasmodium falciparum minimum inhibitory concentration (MIC) data (μg/ml) for compounds of the present invention is provided in Table 4 below. See FIG. 2.

TABLE 4

| | FSM | Compound A | Compound 52 |
|---|---|---|---|
| Run 1 | 1.391 | 0.3413 | 2.395 |
| Run 2 | 0.7607 | 0.5241 | 0.002557 |
| Run 3 | 0.5708 | 90.65 | 9.9 |
| Mean | 0.9075 | 30.50513 | 4.099186 |
| SEM | 0.247888 | 30.07248 | 2.9815 |

### EXAMPLES

### Example 1: (2E)-3-(3,4-Dichlorophenyl) prop-2-enal

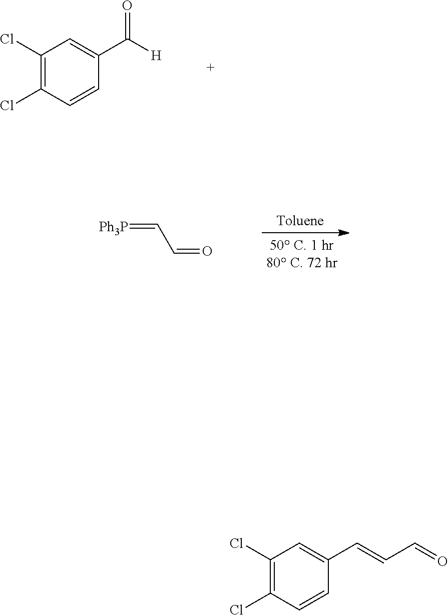

A solution of 3,4-dichlorophenyl benzaldehyde (5.45 g, 31.1 mmol) and triphenylphosphoranylidene acetaldehyde (10.42 g, 34.3 mmol) in 200 mL anhydrous toluene under nitrogen was stirred at 50° C. for 1 hour and at 80° C. for 72 hours. The toluene was removed under reduced pressure. The crude residue was purified via column chromatography (1:1 dichloromethane:hexanes) to afford a yellow solid (4.04 g, 65%). ¹H NMR (400 MHz, CDCl₃) δ (ppm): 6.68 (dd, 1H), 7.37 (d, 1H), 7.45 (m, 3H), 9.71 (d, 1H).

### Example 2: Diethyl [1-(3,4-dichlorophenyl)-3,3-diphenoxypropyl] phosphonate

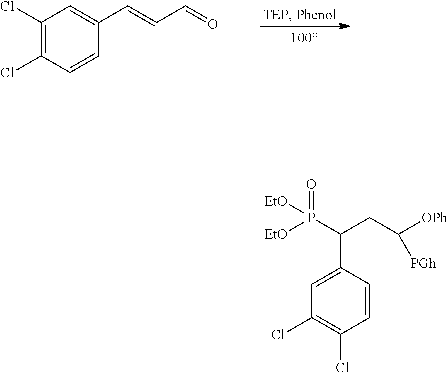

To a flask containing (2E)-3-(3,4-dichlorophenyl) prop-2-enal (4.04 g, 20.1 mmol) were added phenol (4.92 g, 52.2 mmol) and triethylphosphite (4.18 g, 25.1 mmol) under nitrogen. The mixture was subject to an oil bath at 100° C. for 48-72 hours. The excess triethylphosphite was removed under reduced pressure. The crude residue was purified via column chromatography (3:2 hexanes:ethyl acetate) to yield a yellow oil (6.91 g, 67%). ¹H NMR (400 MHz, CDCl₃) δ (ppm): 1.13 (t, 3H), 1.25 (t, 3H), 2.51 (m, 1H), 2.76 (m, 1H), 3.32-3.45 (m, 1H), 3.74-3.87 (m, 1H), 3.89-4.17 (m, 2H), 5.68 (dd, 1H), 6.82-6.93 (m, 3H), 6.95-7.03 (m, 2H), 7.17-7.26 (m, 6H), 7.36-7.47 (m, 2H).

### Example 3: Diethyl [1-(3,4-dichlorophenyl)-3-oxopropyl] phosphonate

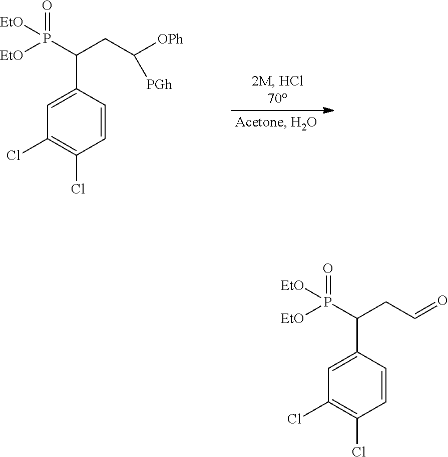

To a solution of diethyl [1-(3,4-dichlorophenyl)-3,3-diphenoxypropyl] phosphonate (3.88 g, 7.62 mmol) in 73 mL acetone were added 8.2 mL of 2M HCl acid and 5.4 mL of water. The mixture was subject to an oil bath at 70° C. for 48 hours. After removing the acetone under reduced pressure, the residue was dissolved in dichloromethane (50 mL) and washed with distilled water (2×50 mL). The organic phase was dried over sodium sulfate and filtered. Dichloromethane was removed under reduced pressure. The crude residue was purified via column chromatography (3:2 dichloromethane:ethyl acetate) to afford a colorless oil as the product (2.02 g, 78%). ¹H NMR (400 MHz, CDCl₃) δ (ppm): 1.11 (t, 3H), 1.23 (t, 3H), 2.99-3.26 (m, 2H), 3.59-3.74 (ddd, 1H), 3.79-3.89 (m, 1H), 3.91-3.99 (m, 2H), 4.00-4.13 (m, 2H), 7.12-7.52 (m, 3H), 9.66 (s, 1H).

### Example 4: O-benzylhydroxylamine

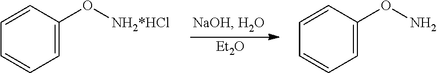

Sodium hydroxide (0.24 g, 5.95 mmol) and O-benzylhydroxylamine hydrochloride (0.95 g, 5.95 mmol) were dissolved in 30 mL of diethyl ether and 10 mL of distilled water. The mixture was stirred for 0.5 hours at room temperature. The aqueous phase was then extracted with diethyl ether (3×50 mL). The organic phase was dried over sodium sulfate and filtered. Diethyl ether was removed under reduced pressure to yield a colorless liquid (0.72 g, quantitative). ¹H NMR (400 MHz, CDCl₃) δ (ppm): 4.67 (s, 2H), 5.09 (bs, 2H), 7.35 (m, 6H).

### Example 5: Diethyl {3-[(benzyloxy) amino]-1-(3,4-dichlorophenyl) propyl}phosphonate

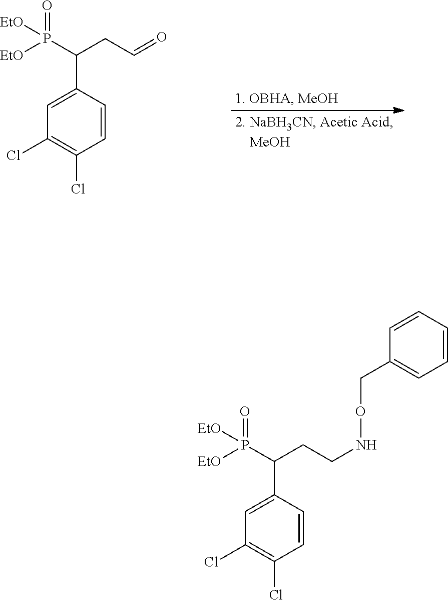 [SMILES: CCOP(=O)(OCC)C(CC=O)c1ccc(Cl)c(Cl)c1 ; CCOP(=O)(OCC)C(CCNOCc1ccccc1)c1ccc(Cl)c(Cl)c1]

To a solution of diethyl [1-(3,4-dichlorophenyl)-3,3-diphenoxypropyl] phosphonate (2.34 g, 6.90 mmol) in 70 mL anhydrous methanol was added O-benzylhydroxylamine (0.93 g, 7.59 mmol) under nitrogen and stirred at room temperature for overnight. To the reaction mixture were added sodium cyanoborohydride (1.52 g, 24.1 mmol) and 1.97 ml of acetic acid. The mixture stirred at room temperature for three hours. The methanol was removed under reduced pressure. The crude residue was dissolved in dichloromethane and washed with saturated NaHCO₃ (60 mL) until the aqueous phase was slightly basic. The aqueous phase was then extracted with dichloromethane (5×50 mL). The combined organic phases were dried over sodium sulfate and filtered. The dichloromethane was removed under reduced pressure. The crude product was purified by via column chromatography (97:3 dichloromethane:methanol) and yielded a colorless oil (1.28 g, 42%). 1H NMR (400 MHz, CDCl₃) δ (ppm): 1.15 (t, 3H), 1.28 (t, 3H, J=7.6 Hz), 1.95-2.13 (m, 1H), 2.27-2.42 (m, 1H), 2.58-2.74 (m, 1H), 2.79-2.90 (m, 1H), 3.11-3.27 (ddd, 1H), 3.76-3.88 (m, 1H), 3.90-3.98 (m, 1H), 4.01-4.12 (m, 2H), 4.54-4.76 (s, 2H), 5.49 (bs, 1H), 7.01-7.20 (m, 1H), 7.23-7.45 (m, 7H).

### Example 6: Diethyl {3-[N-(benzyloxy) acetamido]-1-(3,4-dichlorophenyl) propyl} phosphonate

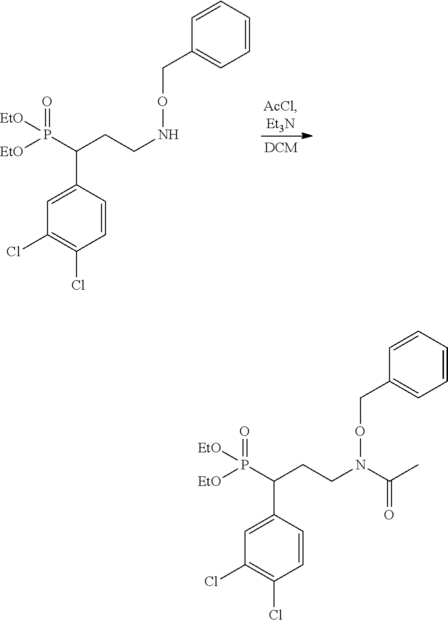 [SMILES: CCOP(=O)(OCC)C(CCNOCc1ccccc1)c1ccc(Cl)c(Cl)c1 ; CCOP(=O)(OCC)C(CCN(OCc1ccccc1)C(C)=O)c1ccc(Cl)c(Cl)c1]

Diethyl {3-[(benzyloxy) amino]-1-(3,4-dichlorophenyl) propyl} phosphonate (1.51 g, 3.93 mmol) was dissolved in 34 mL of anhydrous dichloromethane under nitrogen and cooled to 0° C. Triethylamine (0.686 g, 6.78 mmol) and acetyl chloride (0.319 g, 4.07 mmol) were added to the reaction mixture at 0° C. The mixture was warmed up to room temperature and allowed to stir overnight. The reaction was quenched with water. The aqueous phase was extracted with dichloromethane (3×70 mL). The organic phase was dried over sodium sulfate and filtered. After dichloromethane was removed under reduced pressure, the crude product was purified via column chromatography (100% ethyl acetate). The desired product was obtained as a slightly yellow oil (1.40 g, 85%). ¹H NMR (400 MHz, CDCl₃) δ (ppm): 1.15 (t, 3H), 1.27 (t, 3H), 2.03 (s, 3H), 2.09-2.3 (m, 1H), 2.34-2.50 (m, 1H), 2.93-3.08 (ddd, 1H), 3.39-3.60 (m, 2H), 3.76-3.88 (m, 1H), 3.90-3.98 (m, 1H), 3.99-4.11 (m, 2H), 4.72 (s, 2H), 7.12-7.44 (m, 8H).

### Example 7: Diethyl [1-(3,4-dichlorophenyl)-3-(N-hydroxyacetamido) propyl] phosphonate

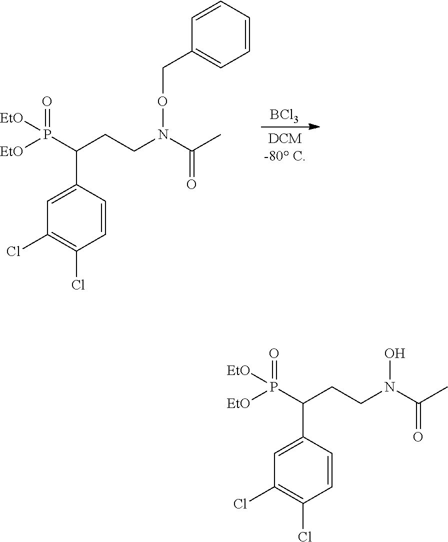

A solution of diethyl {3-[N-(benzyloxy) acetamido]-1-(3,4-dichlorophenyl) propyl} phosphonate (1.37 g, 2.81 mmol) was dissolved in 28 mL of anhydrous dichloromethane under nitrogen and cooled to −80° C. To the solution was added boron trichloride (1M in dichloromethane) (14.5 mL, 14.4 mmol) dropwise. The reaction mixture was stirred at −80° C. for four to five hours and then was quenched with cold saturated NaHCO₃. The aqueous phase was extracted with ethyl acetate (4×60 mL). The organic phases were combined and dried over sodium sulfate and filtered. The ethyl acetate was removed under reduced pressure. The crude product was purified via column chromatography (97:3 dichloromethane:methanol), to afford an orange, viscous oil (1.15 g, 97%). ¹H NMR (400 MHz, CDCl₃) δ (ppm): 1.15-1.36 (m, 6H), 1.87 (m, 1H), 2.08 (s, 3H), 2.43 (m, 1H), 3.12 (m, 1H), 2.79-2.90 (m, 1H), 3.42 (m, 1H), 3.68 (m, 1H), 3.90-4.11 (m, 4H), 7.13-7.24 (m, 1H), 7.38-7.49 (m, 2H), 9.45 (bs, 1H).

### Example 8: Diethyl {3-[N-({[1,1′-biphenyl]-4-yl} methoxy) acetamido]-1-(3,4-dichlorophenyl) propyl} phosphonate

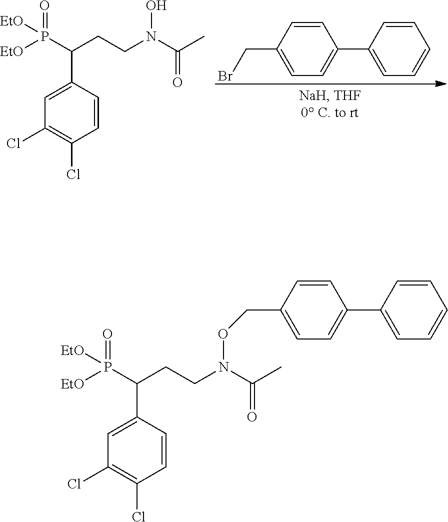

Diethyl [1-(3,4-dichlorophenyl)-3-(N-hydroxyacetamido) propyl] phosphonate (0.098 g, 0.245 mmol) was dissolved in anhydrous tetrahydrofuran (2.45 mL) under a nitrogen atmosphere and cooled to 0° C. Sodium hydride (5.9 mg, 0.245 mmol) was added to the solution and stirred for 20 minutes. To the reaction mixture was added 4-bromomethylbiphenyl (0.061 g, 0.245 mmol). The mixture was warmed to room temperature and stirred for 48 hours. After removing the tetrahydrofuran under reduced pressure, the residue was dissolved in dichloromethane and quenched with water. The aqueous phase was extracted with dichloromethane (5×20 mL). The organic phase was dried over sodium sulfate and filtered. The solvent was removed under reduced pressure. The crude product was purified via column chromatography (95:5 dichloromethane:methanol) to yield a slightly yellow oil (86.9 mg, 63%). ¹H NMR (400 MHz, CDCl₃) δ (ppm): 1.15 (t, 3H), 1.27 (t, 3H), 1.69 (m, 1H), 2.07 (s, 3H), 2.183 (m, 1H), 2.45 (m, 1H), 2.98 (ddd, 1H), 3.43-3.62 (m, 2H), 3.77-3.86 (m, 1H), 3.89-3.97 (m, 1H), 3.98-4.14 (m, 2H), 4.75 (s, 2H), 7.13-7.29 (m, 2H), 7.30-7.51 (m, 6H), 7.55-7.66 (m, 4H).

### Example 9: Diethyl [1-(3,4-dichlorophenyl)-3-(N-{[4-(propan-2-yl) phenyl] methoxy} acetamido) propyl] phosphonate

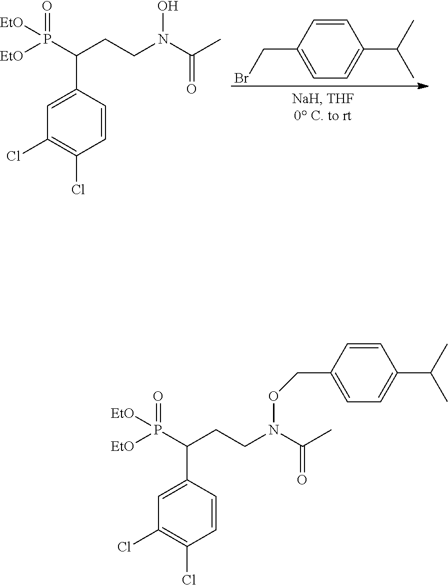

Diethyl [1-(3,4-dichlorophenyl)-3-(N-hydroxyacetamido) propyl] phosphonate (0.130 g, 0.328 mmol) was dissolved in anhydrous tetrahydrofuran (3.30 mL) under a nitrogen atmosphere and cooled to 0° C. Sodium hydride (8.7 mg, 0.361 mmol) was added to the solution and stirred for 20 minutes. To the reaction mixture was added 4-isopropyl-benzylbromide (0.077 g, 0.361 mmol). The mixture was warmed to room temperature and stirred overnight. After removing the tetrahydrofuran under reduced pressure, the residue was dissolved in dichloromethane and quenched with water. The aqueous phase was extracted with dichloromethane (5×20 mL). The organic phase was dried over sodium sulfate and filtered. The solvent was removed under reduced pressure. The crude product was purified via silica gel preparatory plate (97:3 dichloromethane:methanol) to yield a slightly yellow oil (70 mg, 40%). ¹H NMR (400 MHz, CDCl₃) δ (ppm): 1.16 (t, 6H), 1.26 (d, 6H), 1.633 (s, 1H), 2.04 (s, 3H), 2.17 (m, 1H), 2.44 (m, 1H), 2.86-3.09 (m, 1H), 3.40-3.62 (m, 2H), 3.76-3.87 (m, 1H), 3.90-3.98 (m, 1H), 3.98-4.13 (m, 2H), 4.61-4.75 (s, 2H), 6.73-7.09 (m, 3H), 7.09-7.30 (m, 3H), 7.32-7.46 (m, 1H).

### Example 10: Diethyl [1-(3,4-dichlorophenyl)-3-{N-[(naphthalen-2-yl) methoxy] acetamido} propyl] phosphonate

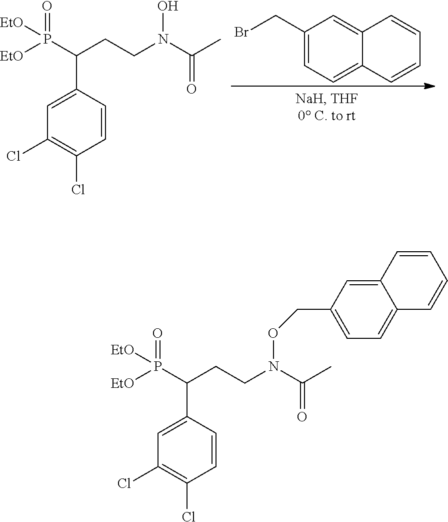

Diethyl [1-(3,4-dichlorophenyl)-3-(N-hydroxyacetamido) propyl] phosphonate 0.111 g, 0.280 mmol) was dissolved in anhydrous tetrahydrofuran (2.80 mL) under a nitrogen atmosphere and cooled to 0° C. Sodium hydride (7.4 mg, 0.307 mmol) was added to the solution and stirred for 20 minutes. To the reaction mixture was added 2-bromomethyl-naphthalene (0.068 g, 0.307 mmol). The mixture was warmed to room temperature and stirred for 48 hours. After removing the tetrahydrofuran under reduced pressure, the residue was dissolved in dichloromethane and quenched with water. The aqueous phase was extracted with dichloromethane (5'20 mL). The organic phase was dried over sodium sulfate and filtered. The solvent was removed under reduced pressure. The crude product was purified via column chromatography (95:5 dichloromethane:methanol) to yield a slightly yellow oil (209.5 mg, 75%). ¹H NMR (400 MHz, CDCl₃) δ (ppm): 1.13 (t, 3H), 1.25 (t, 3H), 2.07 (s, 3H), 2.18 (m, 1H), 2.46 (m, 1H), 2.94-3.08 (m, 1H), 3.41-3.62 (m, 2H), 3.76-3.86 (m, 1H), 3.88-3.96 (m, 1H), 3.97-4.08 (m, 2H), 4.80-4.95 (s, 2H), 7.07-7.16 (m, 1H), 7.23-7.43 (m, 3H), 7.46-7.57 (m, 2H), 7.71-7.89 (m, 4H).

### Example 11: Diethyl (3-{N-[(4-bromophenyl) methoxy] acetamido}-1-(3,4-dichlorophenyl) propyl) phosphonate

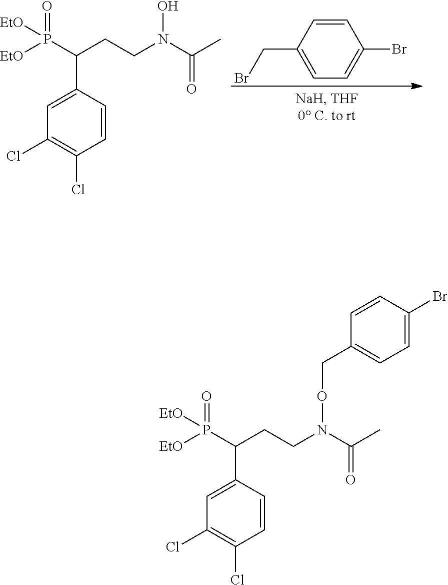

Diethyl [1-(3,4-dichlorophenyl)-3-(N-hydroxyacetamido) propyl] phosphonate (0.470 g, 1.21 mmol) was dissolved in anhydrous tetrahydrofuran (12 mL) under a nitrogen atmosphere and cooled to 0° C. Sodium hydride (0.053 g, 1.33 mmol) was added to the solution and stirred for 20 minutes. To the reaction mixture was added 4-bromobenzyl bromide (0.331 g, 1.33 mmol). The mixture was warmed to room temperature and stirred overnight. After removing the tetrahydrofuran under reduced pressure, the residue was dissolved in dichloromethane and quenched with water. The aqueous phase was extracted with dichloromethane (5×20 mL). The organic phase was dried over sodium sulfate and filtered. The solvent was removed under reduced pressure. The crude product was purified via column chromatography (97:3 dichloromethane:methanol) to yield a slightly yellow oil (0.470 g, 70%). ¹H NMR (400 MHz, CDCl₃) δ (ppm): 1.06-1.41 (m, 6H), 1.59 (s, 1H), 2.02 (m, 3H), 2.17 (m, 1H), 2.43 (m, 1H), 2.91-3.10 (m, 1H), 3.32-3.64 (m, 2H), 3.75-4.20 (m, 4H), 4.65 (s, 2H), 7.02-7.60 (m, 7H).

### Example 12: Diethyl (3-{N-[(4′-methoxy-[1,1′-biphenyl]-4-yl)methoxy]acetamido}-1-(3,4-dichlorophenyl) propyl) phosphonate

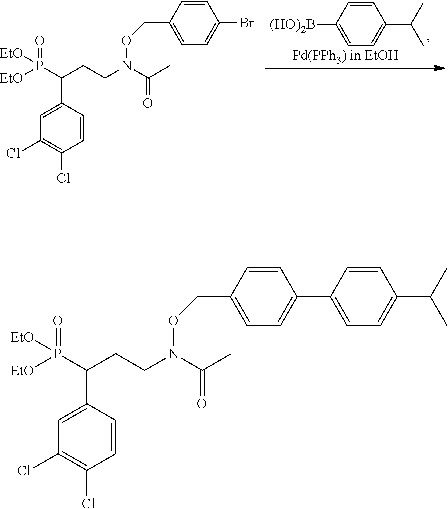 [SMILES: CCOP(=O)(OCC)C(CCN(OCc1ccc(Br)cc1)C(C)=O)c1ccc(Cl)c(Cl)c1 ; CC(C)c1ccc(B(O)O)cc1 ; CCOP(=O)(OCC)C(CCN(OCc1ccc(-c2ccc(C(C)C)cc2)cc1)C(C)=O)c1ccc(Cl)c(Cl)c1]

Diethyl (3-{N-[(4-bromophenyl) methoxy] acetamido}-1-(3,4-dichlorophenyl) propyl) phosphonate (0.310 g, 0.547 mmol) was dissolved in toluene (2.8 mL). To the solution was added Pd(PPh₃)₄ (0.064 g, 0.055 mmol) and stirred at room temperature for 15 minutes. After 15 minutes, to the reaction mixture was added a solution of 4-isopropylphenyl boronic acid (0.448 g, 2.73 mmol) in ethanol (1 mL) and stirred for 15 minutes. To the reaction mixture were added 0.83 mL 2M Na₂CO₃. The reaction mixture was stirred at 75° C. overnight. The mixture was filtered with a membrane filter then the toluene was removed under reduced pressure. The crude residue was dissolved in dichloromethane and washed with distilled water (20 mL). The aqueous phase was extracted with dichloromethane (3×20 mL). The organic phase was dried over sodium sulfate and filtered. Dichloromethane was removed under reduced pressure and the crude product was purified via column (5:1 dichloromethane:ethyl acetate) to afford a yellow oil (64.7 mg, 19%). ¹H NMR (400 MHz, CDCl₃) δ (ppm): 1.147-1.285 (m, 12H), 1.581 (t, 2H), 2.008 (s, 3H), 2.453 (m, 1H), 2.978 (m, 2H), 3.483 (m, 2H), 3.795-4.104 (m, 3H), 4.790 (t, 2H), 7.093-7.738 (m, 11H).

### Example 13: Diethyl [1-(3,4-dichlorophenyl)-3-[N-(4-phenylbutoxy) acetamido] propyl] phosphonate

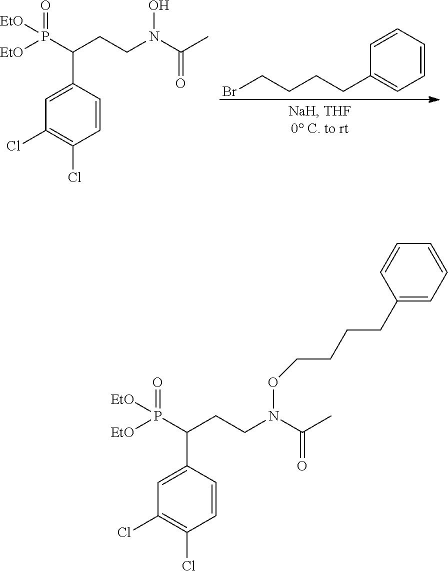 [SMILES: CCOP(=O)(OCC)C(CCN(O)C(C)=O)c1ccc(Cl)c(Cl)c1 ; BrCCCCc1ccccc1 ; CCOP(=O)(OCC)C(CCN(OCCCCc1ccccc1)C(C)=O)c1ccc(Cl)c(Cl)c1]

Diethyl [1-(3,4-dichlorophenyl)-3-(N-hydroxyacetamido) propyl] phosphonate (0.154 g, 0.389 mmol) was dissolved in anhydrous tetrahydrofuran (2.80 mL) under a nitrogen atmosphere and cooled to 0° C. Sodium hydride (0.017 g, 0.428 mmol) was added to the solution and stirred for 20 minutes. To the reaction mixture was added 1-bromo-4-phenyl butane (0.091 g, 0.428 mmol). The mixture was warmed to room temperature and stirred for four days. After removing the tetrahydrofuran under reduced pressure, the residue was dissolved in dichloromethane and quenched with water. The aqueous phase was extracted with dichloromethane (5×20 mL). The organic phase was dried over sodium sulfate and filtered. The solvent was removed under reduced pressure. The crude product was purified via column chromatography (97:3 dichloromethane:methanol) to yield a slightly yellow oil (33.9 mg, 16%). ¹H NMR (400 MHz, CDCl₃) δ (ppm): 1.16 (t, 3H), 1.28 (t, 3H), 1.66 (dq, 5H), 2.02 (s, 3H), 2.16 (m, 1H), 2.40 (1H), 2.56-2.69 (m, 2H), 2.90-3.09 (m, 1H), 3.38-3.57 (m, 2H), 3.62-3.76 (m, 2H), 3.77-3.89 (m, 1H), 3.90-3.98 (m, 1H), 3.99-4.19 (m, 1H), 7.05-7.50 (m, 9H).

### Example 14: Diammonium-[1-(3,4-dichlorophenyl)-3-[N-(1-phenylethoxy) acetamido]propyl phosphonate

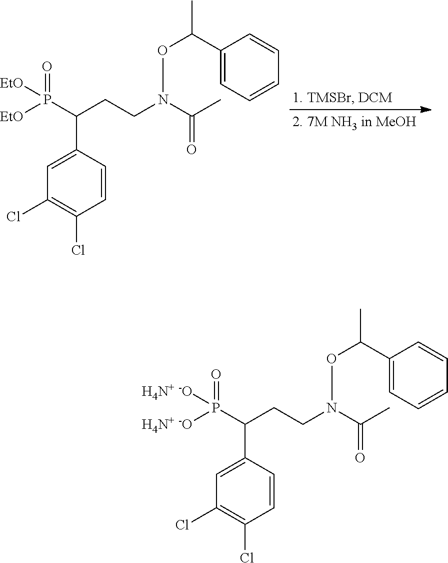

A solution of diethyl [1-(3,4-dichlorophenyl)-3-[N-(1phenylethoxy) acetamido] propyl] phosphonate (48.9 mg, 0.098 mmol) in anhydrous dichloromethane (0.98 mL) under nitrogen was cooled down to 0° C. Trimethylsilyl bromide (0.075 g, 0.49 mmol) was added dropwise to the solution. The mixture was warmed to room temperature and stirred overnight. Trimethylsilyl bromide and dichloromethane were removed under reduced pressure. The crude residue was stirred at room temperature in anhydrous methanol for one to two hours. The methanol was removed under reduced pressure. To the crude residue was added 7M NH₃ in methanol (0.033 mL) and stirred at room temperature for one to two hours. The excess 7M NH₃ in methanol was removed under pressure to yield an orange solid (46 mg, quantitative). ¹H NMR (400 MHz, DMSO-d₆) δ (ppm): 0.78 (d, 3H), 1.73 (d, 2H), 2.43 (s, 3H), 2.98 (m, 1H), 3.17(m, 1H), 3.26-3.51 (m, 1H), 4.66-4.84 (m, 1H), 6.98-7.79 (m, 8H). HRMS (ESI⁻) calculated for C₁₉H₂₈Cl₂N₃O₅P 479.11, found 444 [M-2NH3-1]⁻.

### Example 15: Diammonium-[1-(3,4-Dichlorophenyl)-3-{N-[(naphthalen-2-yl) methoxy]acetamido} propyl]propyl phosphonate

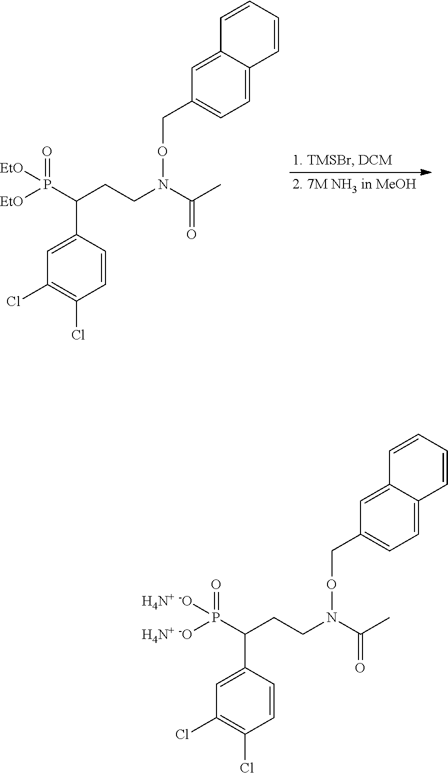

A solution of diethyl [1-(3,4-dichlorophenyl)-3-{N-[(naphthalen-2-yl) methoxy] acetamido} propyl] phosphonate (0.113 g, 0.21 mmol) in anhydrous dichloromethane (2.1 mL) under nitrogen was cooled down to 0° C. Trimethylsilyl bromide (0.324 g, 2.11 mmol) was added dropwise to the solution. The mixture was warmed to room temperature and stirred overnight. Trimethylsilyl bromide and dichloromethane were removed under reduced pressure. The crude residue was stirred at room temperature in anhydrous methanol for one to two hours. The methanol was removed under reduced pressure. To the crude residue was added 7M NH₃ in methanol (0.061 mL) and stirred at room temperature for one to two hours. The excess 7M NH₃ in methanol was removed under pressure to yield an orange solid (107.8 mg, 98%). ₁H NMR (400 MHz, DMSO-d₆) δ (ppm): 0.88 (d, 1H), 1.30 (d, 1H), 2.01 (s, 3H), 2.41 (m, 1H), 2.55 (m, 1H), 2.72-2.87(m, 1H), 3.19-3.41 (m, 1H), 3.47-3.66 (m, 1H), 4.84-5.06 (q, 1H), 7.06-8.05 (m, 10H). HRMS (ESI⁺) calculated for C₂₂H₂₈Cl₂N₃O₅P 515.4, found 482 [M-2NH3+1]⁺.

### Example 16: 1-(1-bromoethyl)-4-methoxybenzene

In a nitrogen atmosphere, to a solution of 1-(4-methoxyphenyl) ethanol (1.05 g, 6.90 mmol) at 0° C. in anhydrous dichloromethane (11.6 mL) was added phosphorous tribromide (2.24 g, 8.28 mmol) dropwise. The mixture was stirred at 0° C. for four to five hours. The mixture was diluted with dichloromethane and washed sequentially with saturated NaHCO₃ (15 mL), distilled water (15 mL), and brine (15 mL). The organic phase was dried over sodium sulfate and filtered to yield a colorless liquid (1.38 g, 93%). ¹H NMR (400 MHz, CDCl₃) δ (ppm): 2.02 (d, 3H), 2.33 (s, 3H), 5.16-5.25 (q, 1H), 7.05-7.50 (m, 4H).

### Example 17: 1-(1-bromoethyl)-4-methylbenzene

In a nitrogen atmosphere, to a solution of 1-(4-methylphenyl) ethanol (1.39 g, 10.21 mmol) at 0° C. in anhydrous dichloromethane (102 mL) was added phosphorous tribromide (3.04 g, 11.23 mmol) dropwise. The mixture was stirred at 0° C. for four to five hours. The mixture was diluted with dichloromethane and washed sequentially with saturated NaHCO₃ (15 mL), distilled water (15 mL), and brine (15 mL). The organic phase was dried over sodium sulfate and filtered to yield a colorless liquid (1.01 g, 75%). ¹H NMR (400 MHz, CDCl₃) δ (ppm): 2.02 (d, 3H), 3.78 (s, 3H), 5.19-5.26 (q, 1H), 7.05-7.50 (m, 4H).

### Example 18: Diethyl [3-{acetyl[1-(4-methoxyphenyl)ethoxy]amino}-1-(3,4-dichlorophenyl)propyl] phosphonate

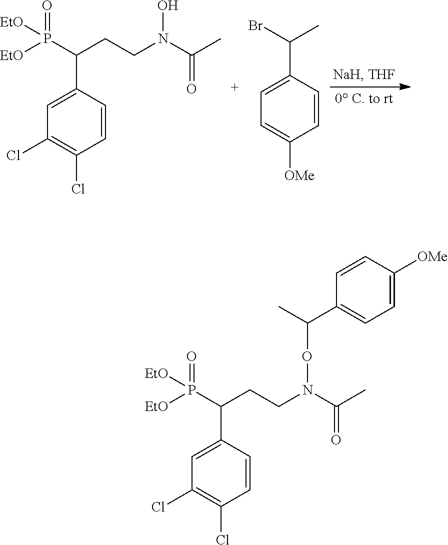

Diethyl [1-(3,4-dichlorophenyl)-3-(N-hydroxyacetamido) propyl] phosphonate (0.205 g, 0.514 mmol) was dissolved in anhydrous tetrahydrofuran (5 mL) under a nitrogen atmosphere and cooled to 0° C. Sodium hydride (0.014 g, 0.565 mmol) was added to the solution and stirred for 20 minutes. To the reaction mixture was added 1-(1-bromoethyl)-4-methoxybenzene (0.122 g, 0.565 mmol). The mixture was warmed to room temperature and stirred for 48 hours. After removing the tetrahydrofuran under reduced pressure, the residue was dissolved in dichloromethane and quenched with water. The aqueous phase was extracted with dichloromethane (5×20 mL). The organic phase was dried over sodium sulfate and filtered. The solvent was removed under reduced pressure. The crude product was purified via column chromatography (99:1 dichloromethane:methanol) to yield a slightly yellow oil (58.2 mg, 21%). ¹H NMR (400 MHz, CDCl₃) δ (ppm): 1.14 (t, 3H), 1.28 (t, 3H), 1.50 (d, 3H), 2.02 (s, 3H), 1.91 (s, 3H), 2.30 (m, 1H), 3.39-3.59 (m, 1H), 3.75-3.87 (d, 4H), 3.88-3.97 (m, 1H), 3.98-4.11 (m, 2H), 4.56-4.79 (m, 1H), 6.73-6.89 (m, 2H), 7.06-7.18 (m, 3H), 7.31-7.44 (m, 2H).

### Example 19: Diethyl [3-{acetyl[1-(4-methylphenyl)ethoxy]amino}-1-(3,4-dichlorophenyl)propyl] phosphonate

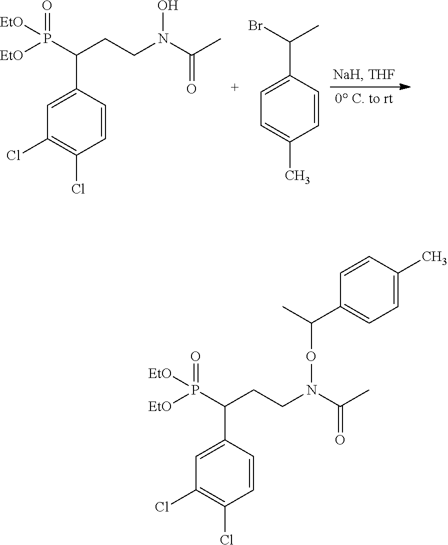

Diethyl [1-(3,4-dichlorophenyl)-3-(N-hydroxyacetamido) propyl] phosphonate (0.226 g, 0.568 mmol) was dissolved in anhydrous tetrahydrofuran (6 mL) under a nitrogen atmosphere and cooled to 0° C. Sodium hydride (0.015 g, 0.625 mmol) was added to the solution and stirred for 20 minutes. To the reaction mixture was added 1-(1-bromoethyl)-4-methylbenzene (0.124 g, 0.625 mmol). The mixture was warmed to room temperature and stirred for 48 hours. After removing the tetrahydrofuran under reduced pressure, the residue was dissolved in dichloromethane and quenched with water. The aqueous phase was extracted with dichloromethane (5×20 mL). The organic phase was dried over sodium sulfate and filtered. The solvent was removed under reduced pressure. The crude product was purified via silica gel preparatory plate chromatography (97:3 dichloromethane:methanol) to yield a slightly yellow oil (0.100 g, 34%). ¹H NMR (400 MHz, CDCl₃) δ (ppm): 1.14 (t, 3H), 1.28 (t, 3H), 1.50 (d, 3H), 2.02 (s, 3H), 1.91 (s, 1H), 2.78-3.01 (m, 1H), 3.39-3.59 (m, 1H), 3.75-3.87 (m, 1H), 3.98-4.11 (m, 2H), 4.56-4.79 (m, 1H), 6.73-6.89 (m, 2H), 7.06-7.18 (m, 3H), 7.31-7.44 (m, 2H).

### Example 20: Diammonium [3-{acetyl[1-(4-methoxyphenyl)ethoxy]amino}-1-(3,4-dichlorophenyl)propyl] phosphonate

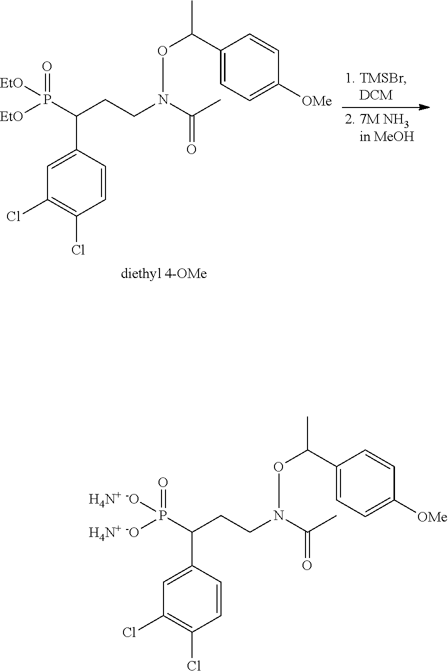

A solution of diethyl 4-OMe (0.047 g, 0.088 mmol) in anhydrous dichloromethane (1 mL) under nitrogen was cooled down to 0° C. Trimethylsilyl bromide (0.041 g, 0.265 mmol) was added dropwise to the solution under nitrogen. The mixture was warmed to room temperature and stirred overnight. Trimethylsilyl bromide and dichloromethane were removed under reduced pressure. The crude residue was stirred at room temperature in anhydrous methanol for one to two hours. The methanol was removed under reduced pressure. To the crude residue was added 7M NH₃ in methanol (0.025 mL) and stirred at room temperature for one to two hours. The excess 7M NH₃ in methanol was removed under pressure to yield an off-white solid (45 mg, quantitative). ¹H NMR (400 MHz, DMSO-d₆) δ (ppm): 0.78 (d, 3H), 1.73 (d, 2H), 2.43 (s, 3H), 2.98 (m, 1H), 3.17(m, 1H), 3.26-3.51 (m, 1H), 4.66-4.84 (m, 1H), 6.98-7.79 (m, 8H). HRMS (ESI⁻) calculated for C₂₀H₃₀Cl₂N₃O₆P 509.12, found 474 [M-2NH3-1]⁻.

### Example 21: Diammonium [3-{acetyl[1-(4-methylphenyl)ethoxy]amino}-1-(3,4-dichlorophenyl)propyl] phosphonate

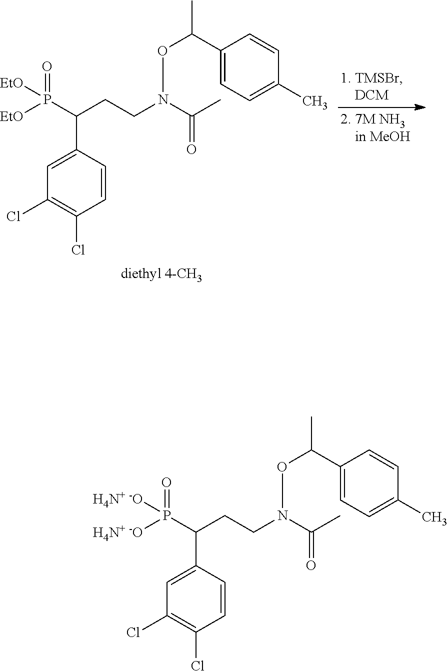

A solution of diethyl 4-CH₃ (0.100 g, 0.194 mmol) in anhydrous dichloromethane (2 mL) under nitrogen was cooled down to 0° C. Trimethylsilyl bromide (0.148 g, 0.968 mmol) was added dropwise to the solution under nitrogen. The mixture was warmed to room temperature and stirred overnight. Trimethylsilyl bromide and dichloromethane were removed under reduced pressure. The crude residue was stirred at room temperature in anhydrous methanol for one to two hours. The methanol was removed under reduced pressure. To the crude residue was added 7M NH₃ in methanol (0.30 mL) and stirred at room temperature for one to two hours. The excess 7M NH₃ in methanol was removed under pressure to yield an off-white solid (95 mg, quantitative). ¹H NMR (400 MHz, DMSO-d₆) δ (ppm): 1.40 (d, 3H), 1.88 (s, 3H), 2.11 (bs, 1H), 2.25 (s, 3H), 2.38-2.47 (m, 1H), 2.48-2.54 (s, 2H), 3.35-3.46 (m, 1H), 4.74-4.84 (m, 1H), 6.95-7.50 (m, 7H). HRMS (ESI⁻) calculated for C₂₀H₃₀Cl₂N₃O₅P 493.13, found 458 [M-2NH3-1]⁻.

### Example 22: ([3-{acetyl[1-(4-methylphenyl)ethoxy]amino}-1-(3,4-dichlorophenyl)propyl])({[(2,2-dimethylpropanoyl) oxy] methoxy}) phosphoryl} oxy) methyl 2,2-dimethylpropanoate

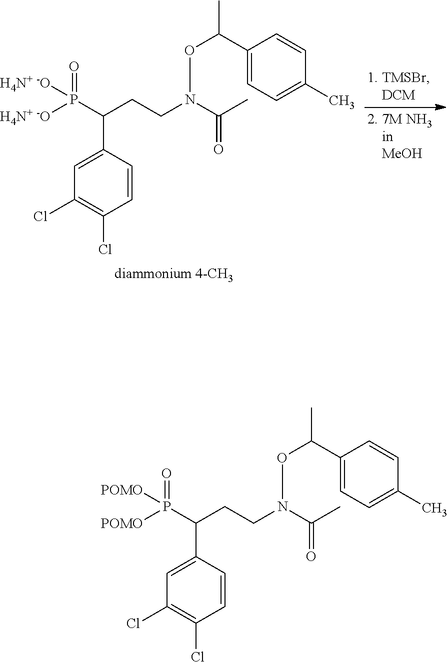

To a solution of diammonium 4-CH₃ (0.080 g, 0.162 mmol) in anhydrous dimethylformamide (1.62 mL) in a nitrogen atmosphere were added N,N-diisopropylethylamine (0.042 g, 0.324 mmol) and pivaloyloxymethyl chloride (0.731 g, 4.86 mmol) at room temperature. The reaction mixture was heated to 60° C. and stirred for 48-72 hours. The dimethylformamide was removed under reduced pressure. The crude residue was dissolved in dichloromethane and washed with saturated NaHCO₃ (10 mL), followed by brine (15 mL). The organic phase was dried over anhydrous sodium sulfate and filtered. The dichloromethane was removed under reduced pressure and the residue was purified via column chromatography (5:1 dichloromethane:ethyl acetate) to yield a slightly yellow oil (64.7 mg, 19%). ¹H NMR (400 MHz, CDCl₃) δ (ppm): 1.12 (s, 9H), 1.16 (s, 9H), 1.97 (m, 3H), 2.13 (s, 1H), 2.37 (s, 1H), 3.01-3.14 (m, 1H), 3.33-3.51 (m, 2H), 4.61-4.71 (m, 2H), 5.39-5.63 (m, 4H), 7.04-7.13 (m, 1H), 7.20-7.26 (m, 2H), 7.28-7.38 (m, 5H).

### Bacterial Strains and Growth Conditions

Recombinant protein was expressed in Escherichia coli Rosetta2(DE3) cells obtained from Novagen (San Diego, CA). E. coli was cultured at 37° C. in Luria-Bertani (LB) media supplemented with 100 μg/mL ampicillin and 34 μg/ml chloramphenicol with constant shaking at 250 rpm. Agar (1.5% wt/vol) was added to prepare solid media.

### Cloning, Expression, and Purification of P. falciparum DXR

The P. falciparum dxr gene was truncated to begin at Lys 75 to remove the apicoplast signaling sequence. A Pf 3D7 trophozoite cDNA library (MRA-297) was acquired from BEi resources and used as the template for amplification of the PIDXR gene. The gene was PCR amplified using primers 5′ CACC AAG AAA CCA ATT AAT GTA GCA 3′ forward and 5′ CTA TAG AGA ATT ATG TTT GTT GTA TAT ATC GGT AG 3′ reverse and cloned into a pET1 00/D-TOPO vector to yield pPIDXR, facilitating the expression of an N-terminal His6-tagged protein.

The expression plasmid (pPIDXR) was separately transformed into chemically competent E. coli Rosetta2(DE3) cells for protein expression. To express the His-tagged protein, a 10 mL overnight seed culture was added to IL of LB media and then incubated with shaking at 37° C. and 250 rpm. At an OD600 of 1.8, protein expression was induced with addition of isopropyl b-D-thiogalactopyranoside (IPTG) to 0.5 mM and the culture was further incubated with shaking at 37° C. and 250 rpm for an additional 18 hours. Cells were harvested via centrifugation (4648×g, 20 min, 4° C.) and stored at −80° C. Protein was subsequently isolated and purified from the cells via chemical lysis and affinity chromatography.

Cells were lysed with lysis buffer A (100 mM Tris pH 8.0, 0.032% lysozyme, 3 mL per gram cell pellet), followed by lysis buffer B (0.1 M CaCh, 0.1 M MgCh, 0.1 M NaCl, 0.020% DNase, 0.3 mL per gram cell pellet). Clarified cell lysate was collected after centrifugation (48,000×g, 20 min, 4° C.) and passed through a TALON immobilized metal affinity column (Clontech Laboratories, Mountain View, CA).

The column was washed with 20 column volumes of Ix equilibrium buffer (50 mM HEPES pH 7.5, 300 mM NaCl), 10 column volumes of Ix wash buffer (50 mM HEPES pH 7.5, 300 mM NaCl, 10 mM imidazole), and 15 column volumes of 2× wash buffer (100 mM HEPES pH 7.5, 600 mM NaCl, 20 mM imidazole). The protein was eluted with 5 column volumes of Ix elution buffer (150 mM imidazole pH 7.0, 300 mM NaCl). Buffer was exchanged with 0.1 M Tris pH 7.5, I mM NaCl, 5 mM DTT during concentration by ultrafiltration. Protein concentration was determined using Advanced Protein Assay Reagent (Cytoskeleton, Denver CO) with y-globulins (Sigma-Aldrich) as the standard. Purified protein was visualized via Coomassie stained SDS-PAGE. The yield of PIDXR averages I mg per IL shake flask.

### P. falciparum Culture

P. falciparum strain 3D7 (wild-type, WT) was obtained through MR4 as part of the BEi Resources Repository, NIAID, NIH (www.mr4.org). A P. falciparum strain containing increased levels of MEP pathway metabolites, had]

(MRA-1257), and its isogenic compliment, had]+Pfl-Iadl-GFP (MRA-1258), were generated in strain 3D7, as reported (Guggisberg et al.). Parasites were cultured in a 2% suspension of human erythrocytes and RPMI 1640 (Sigma) medium supplemented with 27 mM sodium bicarbonate, 11 mM glucose, 5 mM HEPES, I mM sodium pyruvate, 0.37 mM hypoxanthine, 0.01 mM thymidine, 10 μg/mL gentamicin, and 0.5% Albumax (Gibco) at 37° C., 5% 02/5% CO2/90% N2 atmosphere as previously described (Trager et al.; Zhang et al.).

### HepG2 Cell Inhibition Assays

For cytotoxicity assays, HepG2 cells (ATCC HB-8065) were grown in DMEM supplemented with 4 mM L-glutamine (Gibco #11966-025) with either 4.5 g/L D-glucose or 1.8 g/L galactose as carbon source. Cells were trypsinized, resuspended in the respective medium (DMEM/glutamine/glucose or DMEM/glutamine/galactose) to 4×105 cells/mL and 50 μL/well transferred to flat-bottom white opaque tissue culture plates (Falcon #353296) containing 50 L/well of the respective medium with test compound. Compound concentrations were two-fold dilutions ranging from 50 μM to 0.049 μM as well as the drug-free DMSO-only control. All concentrations were tested in duplicate for each carbon source. After 24 h incubation at 5% CO2, 37° C., 10 μL/well of Celltiter-Glo reagent (Promega #G9241) was added and luminescence recorded after 20 min incubation in the dark.

### MEP Pathway Metabolite Assay

Sample preparation. P. falciparum strain 3D7 was cultured at 37° C. in 30 mL volumes in 100 mm tissue culture dishes (Techno Plastic Products) at 4% hematocrit until >8% parasitemia. Cultures were synchronized until >75% of parasites were in ring stage growth, and then treated for 10 h with or without 18a at 65 nM (5× the 3D7 IC50) in triplicate. Cultures were lysed with 5% saponin, the parasite pellets washed with Ix phosphate-buffered saline (PBS), and the pellets stored at −80° C. MEP pathway intermediates were extracted via the addition of glass beads (212-300 u) and 600 μL chilled H2O: chloroform:methanol (3:5:12 v/v) spiked with PIPES (piperazine-N,N′-bis(2-ethanesulfonic acid) as internal standard. The cells were disrupted with the TissueLyser II instrument (Qiagen) using a microcentrifuge tubes adaptor set pre-chilled for 2 min at 20 Hz. The samples were then centrifuged at 16,000 g at 4° C., the supernatants collected, and pellet extraction repeated once more. The supernatants were pooled and 300 μL chloroform and 450 μL of chilled water were added to the supernatants. The tubes were vortexed and centrifuged. The upper layer was transferred to a new tube and dried using a speed-vac. The pellets were re-dissolved in 100 μL of 50% acetonitrile.

LC-MS/MS analysis. For LC separation, a Luna-NH2 column (3 um, 150×2 mm, Phenomenex) was used flowing at 0.4 mL/min. The gradient of the mobile phases A (20 mM ammonium acetate, pH 9.8, 5% ACN) and B (100% acetonitrile) was as follows: 60% B for 1 min, to 6% Bin 3 min, hold at 6% B for 5 min, then back to 60% Bin 0.5 min. The LC system was interfaced with a Sciex QTRAP 6500+ mass spectrometer equipped with a TurbolonSpray (TIS) electrospray ion source. Analyst software (version 1.6.3) was used to control sample acquisition and data analysis. The QTRAP 6500+ mass spectrometer was tuned and calibrated according to the manufacturer's recommendations. Metabolites were detected using MRM transitions that were previously optimized using standards. The instrument was set-up to acquire in negative mode. For quantification, an external standard curve was prepared using a series of standard samples containing different concentrations of metabolites and fixed concentration of the internal standard. The limit of detection for deoxyxylulose 5-phosphate (DOXP), methylerythritol phosphate (MEP), cytidine diphosphate methylerythritol (CDP-ME), and methylerythritol cyclodiphosphate (MEcPP) was 0.0064 μM for a 10 μL injection volume.

Mouse liver microsomes and plasma stability. In this protocol, the metabolic stability of compounds at 1 μM was determined in mouse liver microsomes (MLM) and mouse plasma. For microsomal stability each test compound was incubated in an aqueous reaction mixture consisting of 0.25 μM microsomal protein CYP450 activity, 1.2 mM NADPH, 3.3 mM MgCl2, and 100 mM potassium phosphate buffer (pH 7.4). For plasma stability each test compound was incubated in mouse plasma (VWR). After incubation at 37° C. a 50 μL aliquot of the reaction was transferred to 200 μL ice cold acetonitrile containing internal standard (Enalapril, 100 ng/ml). The quenched reaction mixtures were centrifuged at 3200 rpm for 5 min, and 100 μL of the supernatant were transferred to 96-well plate and analyzed by LC-MS/MS using an Applied Biosystems-Sciex API 4000. Analyte/internal standard peak area ratios were used to evaluate stability. The MRM transitions for enalapril, 12a, and 18a were m/z: 376.9>91.2, 511.197>102.1 and 283.259>102.1, respectively. An Amour C18 column (2.1×30 mm, 5 μm; Analytical Sales and Services, Pompton Plains, NJ) was used for chromatographic separation. Mobile phases were 0.1% formic acid, 1 mM triethylamine in water and acetonitrile with a flow rate of 0.35 mL/min. The starting phase was 0% acetonitrile increased to 100% acetonitrile over 3 minutes. Peak areas were integrated using Analyst Software (AB Sciex, Foster City, CA).

### In Vivo Exposure Study

Animal care and all procedures were conducted at Charles River Laboratories (Wilmington, MA) and performed in accordance with the National Institutes of Health Guide for the Care and Use of Laboratory Animals and approved by the institutional animal care and use committee. Compound 18a was added to 2% methylcellulose 0.5% Tween80 and sonicated to make a 10 mg/mL suspension. The suspension was administered to unfasted female Swiss Weber mice (n=3) at 20 mg/kg i.p. Plasma samples (10 μL) were removed at 0.25, 0.5, 1, 2, 4, 6 and 8 h and stored at −80° C. Plasma samples were added to ice cold acetonitrile containing the internal standard, glafenine, as appropriate to bring samples into the standard curve range (50-10,000 ng/ml), then centrifuged for 5 minutes at 3200 rpm and the supernatant transferred to a 96-well sample plate for analysis by liquid chromatography-tandem mass spectrometry. The MRM transitions for glafenine and 12a were m/z: 370.9>296.9 and 180.075>119.9, respectively. A Synergi 4 μm Hydro-RP column (250×4.6 mm, 80 A) was used for chromatographic separation. Mobile phases were 0.1% formic acid in water and acetonitrile with a flow rate of 1.2 mL/min. The starting phase was 0% acetonitrile for 3 minutes, increased to 60% acetonitrile over 6 minutes. Peak areas were integrated using Analyst Software (AB Sciex, Foster City, CA).

The description of the present embodiments of the invention has been presented for purposes of illustration, but is not intended to be exhaustive or to limit the invention to the form disclosed. Many modifications and variations will be apparent to those of ordinary skill in the art. As such, while the present invention has been disclosed in connection with an embodiment thereof, it should be understood that other embodiments may fall within the spirit and scope of the invention. Patents and publications cited herein are incorporated by reference in their entirety.

1. A compound of formula (I) 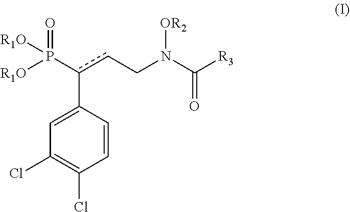
or a tautomer thereof, stereoisomer thereof, prodrug thereof, or pharmaceutically acceptable salt thereof,
wherein
---- represents a bond or is absent;
each R₁ is, independently, —NH₄, —N(alkyl)₄, —N(aryl)₄, C₁₋₄ alkyl, or —(CR^{a}R^{b})ₘ—O(C═O)—C₁₋₆ alkyl, wherein the atom at the left is attached to the oxygen atom;
R₂ is H, —(CR^{c}R^{d})ₙ-aryl, or —(CR^{c}R^{d})ₙ-heteroaryl, wherein the atom at the left is attached to the oxygen atom;
R₃ is H, C₁₋₆ alkyl, C₁₋₆ haloalkyl, C₃₋₆ cycloalkyl, C₁₋₆ alkoxy, C₁₋₆ haloalkoxy, C₃₋₆ cycloalkoxy, —(CR_{c}R_{d})ₚ-aryl or or —(CR^{c}R^{d})ₙ-heteroaryl;
each of R^{a}, R^{b}, R^{c}, and R^{d} is independently H, halogen, or C₁₋₄ alkyl;
each of m and n is independently 1, 2, 3, or 4; and
p is 0, 1, 2, 3, or 4;
wherein each aryl or heteroaryl is, independently, optionally substituted with up to five R⁴ selected from the group consisting of halogen, hydroxyl, cyano, amino, (C₁₋₆ alkyl)amino, di(C₁₋₆ alkyl)amino, C₁₋₆ alkyl, C₁₋₆ haloalkyl, C₃₋₆ cycloalkyl, C₁₋₆ alkoxy, C₁₋₆ haloalkoxy, C₃₋₆ cycloalkoxy, arylalkyl and heteroaryl;
with the proviso that when ---- is absent, each R₁ is ethyl or each R₁ is —CH₂—O—C(═O)—C(CH₃)₃, and R₃ is methyl, then R₂ is not 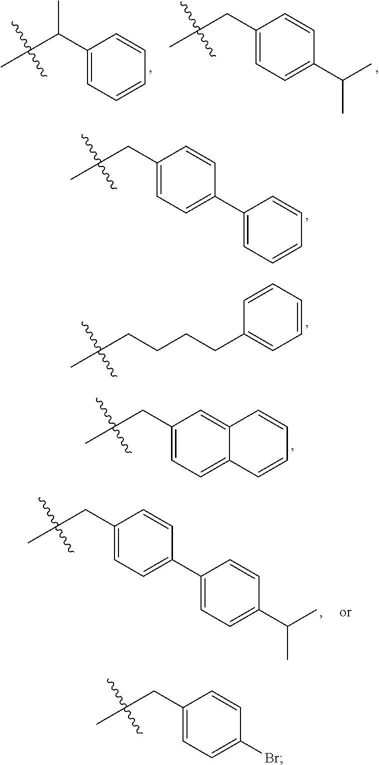
where the squiggly line ( [symbol] ) represents the point of attachment of R₂ to the reset of the molecule.

2. (canceled)

3. A compound of formula (I) 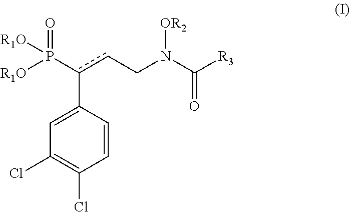
or a tautomer thereof, stereoisomer thereof, prodrug thereof, or pharmaceutically acceptable salt thereof,
wherein
---- represents a bond or is absent;
each R₁ is, independently, —NH₄, —N(alkyl)₄, —N(aryl)₄, C₁₋₄ alkyl, or —(CR^{a}R^{b})ₘ—O(C═O)—C₁₋₆ alkyl, wherein the atom at the left is attached to the oxygen atom;
R₂ is H, —(CR^{c}R^{d})ₙ-aryl, or —(CR^{c}R^{d})ₙ-heteroaryl, wherein the atom at the left is attached to the oxygen atom;
R₃ is CH₃;
each of R^{a}, R^{b}, R^{c}, and R^{d} is independently H, halogen, or C₁₋₄ alkyl;
each of m and n is independently 1, 2, 3, or 4; and
p is 0, 1, 2, 3, or 4;
wherein each aryl or heteroaryl is, independently, optionally substituted with up to five R⁴ selected from the group consisting of halogen, hydroxyl, cyano, amino, (C₁₋₆ alkyl)amino, di(C₁₋₆ alkyl)amino, C₁₋₆ alkyl, C₁₋₆ haloalkyl, C₃₋₆ cycloalkyl, C₁₋₆ alkoxy, C₁₋₆ haloalkoxy, C₃₋₆ cycloalkoxy, arylalkyl and heteroaryl;
with the proviso that the compound of formula (I) is not 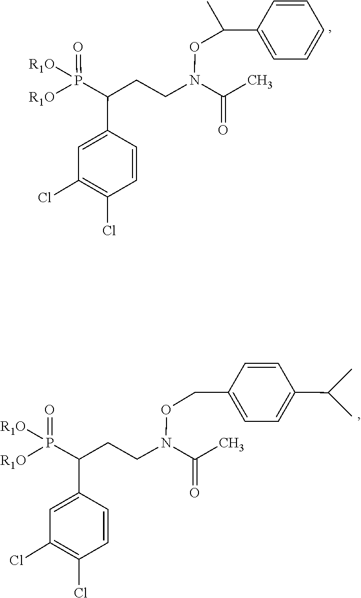 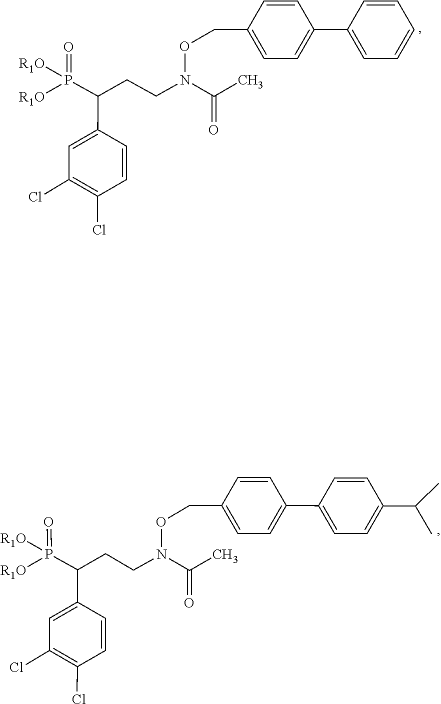 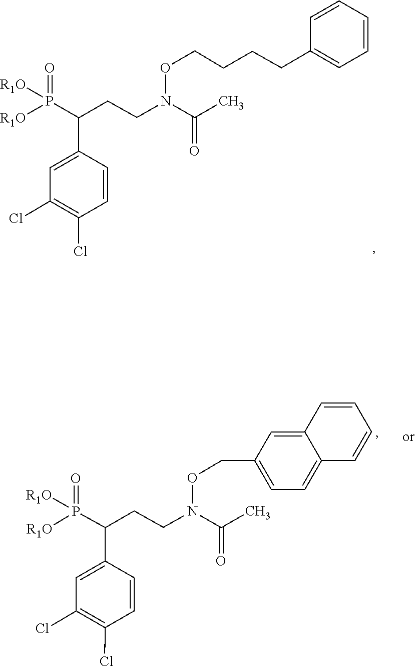 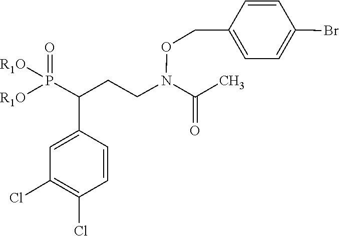
wherein each R₁ is ethyl or each R₁ is —CH₂—O(C═O)—C(CH₃)₃ (pivaloyloxymethyl, POM).

4. The compound according to claim 3, wherein the compound is a mono-salt, a di-salt, or a di-NH₄ salt.

5-6. (canceled)

7. The compound according to claim 3, wherein each R₁ is NH₄.

8. The compound according to claim 3, wherein each R₁ is C₁₋₄ alkyl.

9. The compound according to claim 3, wherein each R₁ is ethyl.

10. The compound according to claim 3, wherein each R₁ is —(CR^{a}R^{b})m-O(C═O)—C₁₋₆ alkyl.

11. The compound according to claim 3, wherein each R₁ is —CH₂O(C═O)—C₁₋₆ alkyl.

12. The compound according to claim 3, wherein each R₁ is —CH₂—O(C═O)—C(CH₃)₃ (pivaloyloxymethyl, POM).

13. The compound according to claim 3, wherein R₂ is H.

14. The compound according to claim 3, wherein R₂ is H, CH(CH₃)Ph, CH₂(4-biphenyl), CH₂(2-napthyl), CH₂(4-iPr4-biphenyl), CH₂CH₂CH₂CH₂phenyl, CH(CH₃)(4-chlorophenyl), CH(CH₃)(4-bromophenyl), CH(CH₃)(4-flurophenyl), CH(CH₃)(4-methoxyphenyl), CH(CH₃)(4-trifluromethylphenyl), or CH(CH₃)(4-methylphenyl).

15. The compound according to claim 3, wherein n is 1, 2, or 3.

16. The compound according to claim 3, wherein n is 1 or 2.

17-19. (canceled)

20. The compound according to claim 1, wherein R₃ is —CH₃.

21. A compound s-according to claim 3, wherein the compound is elected from Tables 1 and 2.

22. A pharmaceutical composition comprising the compound of claim 3 and a pharmaceutically acceptable excipient.

23-26. (canceled)

27. A method for treating or preventing a pathogen in a subject in need thereof comprising administering to the subject an effective amount of a compound of claim 3.

28. The method of claim 27, wherein the pathogen is a Plasmodium parasite species, a Mycobacterium bacteria species, S. aureus, or an ESKAPE pathogen.

29. The method of claim 28, were the pathogen is Plasmodium falciparum or Mycobacterium tuberculosis.

30. The method of claim 27, wherein the subject is a human.
